SEQUENCE LISTING (94 sequences)

SEQ ID NO: 1 - RNA, 20 nt, Artificial
  h17DYSF1
gcuugacagcaccugcaggc

SEQ ID NO: 2 - RNA, 20 nt, Artificial
  h17DYSF2
aggcuuucgaaggcuugaca

SEQ ID NO: 3 - RNA, 20 nt, Artificial
  h18DYSF1
cauagagguugauguagcag

SEQ ID NO: 4 - RNA, 20 nt, Artificial
  h18DYSF2
ggucugggaagccugugaac

SEQ ID NO: 5 - RNA, 20 nt, Artificial
  h19DYSF1
gaagccggccacgauaagcc

SEQ ID NO: 6 - RNA, 20 nt, Artificial
  h19DYSF2
ccuucuguucacugugcucc

SEQ ID NO: 7 - RNA, 20 nt, Artificial
  h20DYSF1
uggcaucauccacauccugc

SEQ ID NO: 8 - RNA, 20 nt, Artificial
  h20DYSF2
ggucaugucgaacuuguucc

SEQ ID NO: 9 - RNA, 20 nt, Artificial
  h20DYSF3
ggcaggucaugucgaacuug

SEQ ID NO: 10 - RNA, 18 nt, Artificial
  h21DYSF1
accaccacagguuucacg

SEQ ID NO: 11 - RNA, 20 nt, Artificial
  h21DYSF2
gcagcugguucugagucucg

SEQ ID NO: 12 - RNA, 21 nt, Artificial
  h24DYSF1
gcauccagaugacgauguccg

SEQ ID NO: 13 - RNA, 20 nt, Artificial
  h24DYSF2
gcuucccacaauucuugcca

SEQ ID NO: 14 - RNA, 20 nt, Artificial
  h30DYSF1
ccgucuucuccaguggcucc

SEQ ID NO: 15 - RNA, 21 nt, Artificial
  h30DYSF2
cggcggaaggcaucugucuug

SEQ ID NO: 16 - RNA, 20 nt, Artificial
  h31DYSF1
uggaaucuucacucuuguca

SEQ ID NO: 17 - RNA, 20 nt, Artificial
  h31DYSF2
ucgugggucuguucacaccg

SEQ ID NO: 18 - RNA, 17 nt, Artificial
  h32DYSF2
gcguagaugguagcggu

SEQ ID NO: 19 - RNA, 20 nt, Artificial
  h32DYSF3
gaguccuuguccaucgcagc

SEQ ID NO: 20 - RNA, 21 nt, Artificial
  h34DYSF2b
uccguuccagacucgguucac

SEQ ID NO: 21 - RNA, 20 nt, Artificial
  h33DYSF1
guguucuucaccaccaccgu

SEQ ID NO: 22 - RNA, 19 nt, Artificial
  h33DYSF2
ggcgguugcucagcaacug

SEQ ID NO: 23 - RNA, 21 nt, Artificial
  h33DYSF3
cuucaccaccaccgucuucug

SEQ ID NO: 24 - RNA, 22 nt, Artificial
  h34DYSF1
cgacggcuggcugccccucguc

SEQ ID NO: 25 - RNA, 20 nt, Artificial
  h32DYSF1b
gcagcguagaugguagcggu

SEQ ID NO: 26 - RNA, 20 nt, Artificial
  h35DYSF1
caaaaccaggaauauggugg

SEQ ID NO: 27 - RNA, 20 nt, Artificial
  h36DYSF1
cauccaggauccuugauguc

SEQ ID NO: 28 - RNA, 20 nt, Artificial
  h42DYSF1
ggccuccacauucuccagcu

SEQ ID NO: 29 - RNA, 20 nt, Artificial
  h42DYSF2
ugucuccuccugcgucuugc

SEQ ID NO: 30 - RNA, 20 nt, Artificial
  h42DYSF3
uggaucuucugucuccuccu

SEQ ID NO: 31 - RNA, 20 nt, Artificial
  h54DYSF1
aacuucauggucuuguaugg

SEQ ID NO: 32 - RNA, 20 nt, Artificial
  h54DYSF2
gaugaagauggccaggaaca

SEQ ID NO: 33 - RNA, 20 nt, Artificial
  h53DYSF1
cugcuacaaucuccaagguc

SEQ ID NO: 34 - RNA, 19 nt, Artificial
  h53DYSF2
aggccgcuccucaugcuca

SEQ ID NO: 35 - RNA, 20 nt, Artificial
  h43DYSF1
ggccuccacauucuccagcu

SEQ ID NO: 36 - RNA, 20 nt, Artificial
  h43DYSF2
ugucuccuccugcgucuugc

SEQ ID NO: 37 - RNA, 20 nt, Artificial
  h43DYSF3
uggaucuucugucuccuccu

SEQ ID NO: 38 - RNA, 21 nt, Artificial
  h20DYSF4
uacuugcgccuccuaagguac

SEQ ID NO: 39 - RNA, 20 nt, Artificial
  h20DYSF5
uacuugcgccuccuaaggua

SEQ ID NO: 40 - RNA, 19 nt, Artificial
  h20DYSF6
uugcgccuccuaagguacu

SEQ ID NO: 41 - RNA, 19 nt, Artificial
  h20DYSF7
augguggcugaguagaagg

SEQ ID NO: 42 - RNA, 20 nt, Artificial
  h20DYSF8
gauggcaucauccacauccu

SEQ ID NO: 43 - RNA, 20 nt, Artificial
  h20DYSF9
augcugaccucaaacuggau

SEQ ID NO: 44 - RNA, 20 nt, Artificial
  h20DYSF10
ucgaacuuguucccguaguu

SEQ ID NO: 45 - RNA, 21 nt, Artificial
  h20DYSF11
ucgaacuuguucccguaguuc

SEQ ID NO: 46 - RNA, 21 nt, Artificial
  h20DYSF12
ucaugucgaacuuguucccgu

SEQ ID NO: 47 - RNA, 20 nt, Artificial
  h21DYSF3
gguagguaguaguaguggca

SEQ ID NO: 48 - RNA, 20 nt, Artificial
  h21DYSF4
acagguuucacguuacccca

SEQ ID NO: 49 - RNA, 19 nt, Artificial
  h21DYSF5
ugauguccucccaguagga

SEQ ID NO: 50 - RNA, 21 nt, Artificial
  h21DYSF6
gauucuauggcugauguccuc

SEQ ID NO: 51 - RNA, 21 nt, Artificial
  h21DYSF7
cucgauucuauggcugauguc

SEQ ID NO: 52 - RNA, 20 nt, Artificial
  h21DYSF8
cugguucugagucucgauuc

SEQ ID NO: 53 - RNA, 21 nt, Artificial
  h21DYSF9
agcaaucccaagcagcugguu

SEQ ID NO: 54 - RNA, 20 nt, Artificial
  h21DYSF10
caccacagguuucacguuac

SEQ ID NO: 55 - DNA, 20 bp, Artificial
  target H18DYSF 1
ctgctacatcaacctctatg

SEQ ID NO: 56 - DNA, 20 bp, Artificial
  target H18DYSF 2
gttcacaggcttcccagacc

SEQ ID NO: 57 - DNA, 20 bp, Artificial
  target H19DYSF1
ggcttatcgtggccggcttc

SEQ ID NO: 58 - DNA, 20 bp, Artificial
  target H19DYSF2
ggagcacagtgaacagaagg

SEQ ID NO: 59 - DNA, 20 bp, Artificial
  target H20DYSF1
gcaggatgtggatgatgcca

SEQ ID NO: 60 - DNA, 20 bp, Artificial
  target H20DYSF2
ggaacaagttcgacatgacc

SEQ ID NO: 61 - DNA, 20 bp, Artificial
  target H20DYSF3
caagttcgacatgacctgcc

SEQ ID NO: 62 - DNA, 18 bp, Artificial
  target H21DYSF1
cgtgaaacctgtggtggt

SEQ ID NO: 63 - DNA, 20 bp, Artificial
  target H21DYSF2
cgagactcagaaccagctgc

SEQ ID NO: 64 - DNA, 21 bp, Artificial
  target H24DYSF1
cggacatcgtcatctggatgc

SEQ ID NO: 65 - DNA, 20 bp, Artificial
  target H24DYSF2
tggcaagaattgtgggaagc

SEQ ID NO: 66 - DNA, 20 bp, Artificial
  target H30DYSF1
ggagccactggagaagacgg

SEQ ID NO: 67 - DNA, 21 bp, Artificial
  target H30DYSF2
caagacagatgccttccgccg

SEQ ID NO: 68 - DNA, 20 bp, Artificial
  target H31DYSF1
tgacaagagtgaagattcca

SEQ ID NO: 69 - DNA, 20 bp, Artificial
  target H31DYSF2
cggtgtgaacagacccacga

SEQ ID NO: 70 - DNA, 20 bp, Artificial
  target H32DYSF1b
accgctaccatctacgctgc

SEQ ID NO: 71 - DNA, 17 bp, Artificial
  target H32DYSF2
accgctaccatctacgc

SEQ ID NO: 72 - DNA, 20 bp, Artificial
  target H32DYSF3
gctgcgatggacaaggactc

SEQ ID NO: 73 - DNA, 22 bp, Artificial
  target H34DYSF1
gacgaggggcagccagccgtcg

SEQ ID NO: 74 - DNA, 21 bp, Artificial
  target H34DYSF2b
gtgaaccgagtctggaacgga

SEQ ID NO: 75 - DNA, 20 bp, Artificial
  target H43DYSF1
agctggagaatgtggaggcc

SEQ ID NO: 76 - DNA, 20 bp, Artificial
  target H43DYSF2
gcaagacgcaggaggagaca

SEQ ID NO: 77 - DNA, 20 bp, Artificial
  target H43DYSF3
aggaggagacagaagatcca

SEQ ID NO: 78 - DNA, 21 bp, Artificial
  target H20DYSF4
gtaccttaggaggcgcaagta

SEQ ID NO: 79 - DNA, 20 bp, Artificial
  target H20DYSF5
taccttaggaggcgcaagta

SEQ ID NO: 80 - DNA, 19 bp, Artificial
  target H20DYSF6
agtaccttaggaggcgcaa

SEQ ID NO: 81 - DNA, 19 bp, Artificial
  target H20DYSF7
ccttctactcagccaccat

SEQ ID NO: 82 - DNA, 20 bp, Artificial
  target H20DYSF8
aggatgtggatgatgccatc

SEQ ID NO: 83 - DNA, 20 bp, Artificial
  target H20DYSF9
atccagtttgaggtcagcat

SEQ ID NO: 84 - DNA, 20 bp, Artificial
  target H20DYSF10
aactacgggaacaagttcga

SEQ ID NO: 85 - DNA, 21 bp, Artificial
  target H20DYSF11
gaactacgggaacaagttcga

SEQ ID NO: 86 - DNA, 21 bp, Artificial
  target H20DYSF12
acgggaacaagttcgacatga

SEQ ID NO: 87 - DNA, 20 bp, Artificial
  target H21DYSF3
tgccactactactacctacc

SEQ ID NO: 88 - DNA, 20 bp, Artificial
  target H21DYSF4
tggggtaacgtgaaacctgt

SEQ ID NO: 89 - DNA, 19 bp, Artificial
  target H21DYSF5
tcctactgggaggacatca

SEQ ID NO: 90 - DNA, 21 bp, Artificial
  target H21DYSF6
gaggacatcagccatagaatc

SEQ ID NO: 91 - DNA, 21 bp, Artificial
  target H21DYSF7
gacatcagccatagaatcgag

SEQ ID NO: 92 - DNA, 20 bp, Artificial
  target H21DYSF8
gaatcgagactcagaaccag

SEQ ID NO: 93 - DNA, 21 bp, Artificial
  target H21DYSF9
aaccagctgcttgggattgct

SEQ ID NO: 94 - DNA, 20 bp, Artificial
  target H21DYSF10
gtaacgtgaaacctgtggtg

(19) United States
(12) Patent Application Publication
(10) Pub. No.: US 2012/0270930 A1
(43) Pub. Date: Oct. 25, 2012

(54) METHODS AND COMPOSITIONS FOR DYSFERLIN EXON-SKIPPING

(75) Inventors: Silvère Maria Van Der Maarel, Oegstgeest (NL); Garrit-Jan B. Van Ommen, Amsterdam (NL); Annemieke Aartsma-Rus, Hoofddorp (NL); Isabella Houweling-Gazzoli, Zoetermeer (NL); Johannes T. Den Dunnen, Rotterdam (NL)

(73) Assignee: Academisch Ziekenhuis Leiden H.O.D.N. Lumc

(21) Appl. No.: 13/504,907

(22) PCT Filed: Oct. 29, 2009
(86) PCT No.: PCT/NL10/50726

§ 371 Date: Jul. 5, 2012

(30) Foreign Application Priority Data
Oct. 29, 2009 (EP) 09174543.0

(51) Int. Cl. A61K 31/7088; C12N 5/071; C07H 21/02; C12Q 1/68

(52) U.S. Cl. 514/44 R; 435/6.12; 435/325; 536/23.1

(57) ABSTRACT

The disclosure provides methods and compositions for inducing exon-skipping in a dysferlin pre-mRNA useful, e.g., in restoring function in a dysferlin deficiency. The disclosure also provides improved methods and compositions for generally inducing exon-skipping in a pre-mRNA.

### CROSS-REFERENCE TO RELATED APPLICATION(S)

This is a national phase entry under 35 U.S.C. §371 of International Patent Application PCT/NL2010/050726, filed Oct. 29, 2010, published in English as International Patent Publication WO 2011/053144 A2 on May 5, 2011, which claims the benefit under Article 8 of the Patent Cooperation Treaty to European Patent Application Ser. No. 09174543.0, filed Oct. 29, 2009.

### TECHNICAL FIELD

The disclosure relates generally to biotechnology and medicine, and provides methods and compositions for inducing exon-skipping in a dysferlin pre-mRNA useful, e.g., in restoring function in a dysferlin deficiency. The disclosure also provides improved methods and compositions for generally inducing exon-skipping in a pre-mRNA.

### BACKGROUND

Muscular dystrophy represents a family of inherited diseases of the muscles. Symptoms may include clumsy movement, difficulty climbing stairs, frequent trips and falls, unable to jump or hop normally, tip toe walking, leg pain, facial weakness, inability to close eyes or whistle, and shoulder and arm weakness. Some forms affect children (e.g., Duchenne dystrophy) and are lethal within two to three decades. Other forms present in adult life and are more slowly progressive. The genes for several dystrophies have been identified, including Duchenne dystrophy (caused by mutations in the dystrophin gene) and the teenage and adult onset Miyoshi dystrophy or its variant, limb girdle dystrophy 2B or LGMD-2B (caused by mutations in the dysferlin gene). These are “loss of function” mutations that prevent expression of the relevant protein in muscle and thereby cause muscle dysfunction.

Dysferlin is a 230-kDa membrane-spanning protein consisting of a single C-terminal transmembrane domain and six C2 domains (Anderson et al. 1999, Hum. Mol. Genet. 8:855-861). In normal muscle, sarcolemma injuries lead to accumulation of dysferlin-enriched membrane patches and resealing of the membrane in the presence of Ca²⁺. Dysferlin deficiency results in defective membrane repair mechanisms (Bansal et al., 2003, Nature 423:168-172; Lennon et al., 2003, J. Biol. Chem. 278:50466-50473). An impaired interaction between dysferlin and annexins A1 and A2 has been discussed as a possible mechanism (Lennon et al., 2003, J. Biol. Chem. 278:50466-5047). Although dysferlin is expressed in human skeletal and cardiac muscles (Anderson et al., 1999, Hum. Mol. Genet. 8:855-861), mutations in the encoding gene (DYSF) lead only to skeletal muscle phenotypes without myocardial involvement, namely limb girdle muscular dystrophy 2B (LGMD2B) and Miyoshi myopathy (Liu et al., 1998, Nat. Genet. 20:31-36).

As there is currently no treatment for the “dysferlinopathies,” lack of dysferlin leads to progressive loss of tissue and function of the muscles of the limbs and girdle (Bansal D. and K. P. Campbell, 2004, Dysferlin and the plasma membrane repair in muscular dystrophy. Trends Cell Biol. 14:206-213). The goal of present treatment is to prevent deformity and allow the patient to function as independently as possible. Consequently, a long-felt need exists for new approaches and better methods to control muscular dystrophy associated with dysferlin deficiency.

### SUMMARY OF THE DISCLOSURE

The present disclosure broadly relates to methods and compositions for exon-skipping in a pre-mRNA.

In one aspect, the disclosure provides a method for providing a cell with an alternatively spliced dysferlin mRNA, the method comprising: a) providing a cell that expresses a dysferlin pre-mRNA with one or more oligonucleotides, in particular, antisense oligonucleotides, for skipping exon(s) (2, 3, 4 and 5), (3 and 4), (5 and 6), 7, 8, 9, (10 and 11), (12 and 13), (14, 15 and 16), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52) or (53 and 54) or a combination thereof, and b) allowing splicing of the pre-mRNA. In one aspect, the disclosure provides a method for providing a cell with an alternatively spliced dysferlin mRNA, the method comprising: a) providing a cell that expresses a dysferlin pre-mRNA with one or more oligonucleotides, in particular, antisense oligonucleotides, for skipping exon(s) (2, 3, 4 and 5), (3 and 4), (5 and 6), 7, 8, 9, (10 and 11), (12 and 13), 14, (15, 16, 17 and 18) 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51 52 and 53), (51 and 52) or (53 and 54) or a combination thereof, and b) allowing splicing of the pre-mRNA. Preferably, the one or more antisense oligonucleotides are provided for skipping exon(s) 32, 34, 36, 42, (20 and 21), (53 and 54), (31, 32 and 33), or a combination thereof Preferably, the one or more antisense oligonucleotides are provided for skipping exon(s) 32, 34, 36, 42, or (20 and 21). Preferably, the one or more antisense oligonucleotides are provided for skipping exon(s) 32, 34, (20 and 21), 24, 30, 41, 42, (5 and 6), (12 and 13), (26 and 27), (28 and 29), 35, 36, 19, or 43. More preferably, one or more antisense oligonucleotides are provided for skipping exon(s) (24, 30, 32, or 34), (30, 32 or 34), or 32 or 34. In some embodiments, exon 17, 32, 34, 35, 36, 41, 42, or a combination thereof, is skipped. In some embodiments, exon 24, 30, or a combination thereof, is skipped. In some embodiments, exon 32, 36 and 42, or a combination thereof, is skipped. In some embodiments, exon 32 and/or 36 is skipped. In some embodiments, only a single dysferlin exon is skipped. In some embodiments, the oligonucleotide comprises a sequence selected from SEQ ID NOS:1-19 and 21-34. In some embodiments, the oligonucleotide comprises a sequence selected from SEQ ID NOS:18 and 19. Preferably, the oligonucleotide comprises a sequence selected from SEQ ID NOS:1-54, more preferably, from SEQ ID NOS:19, 20, 6, 9, 12-15, 24, 25, 35, and 37.

In one aspect, the disclosure provides oligonucleotides or sets of oligonucleotides comprising between 15 and 40 nucleotides complementary to a dysferlin pre-mRNA to induce skipping exon(s) (2, 3, 4 and 5), (3 and 4), (5 and 6), 7, 8, 9, (10 and 11), (12 and 13), (14, 15 and 16), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51 52 and 53), (51 and 52) or (53 and 54) or a combination thereof. In one aspect, the disclosure provides oligonucleotides or sets of oligonucleotides comprising between 15 and 40 nucleotides complementary to a dysferlin pre-mRNA to induce skipping exon(s) (2, 3, 4 and 5), (3 and 4), (5 and 6), 7, 8, 9, (10 and 11), (12 and 13), 14, (15, 16, 17 and 18), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52) or (53 and 54) or a combination thereof. Preferably, the oligonucleotides induce skipping exon(s) 32, 34, 36, 42, (20 and 21), (53 and 54), (31, 32 and 33), or a combination thereof. Preferably, oligonucleotides induce skipping exon(s) 32, 34, 36, 42, or (20 and 21). Preferably, the oligonucleotides induce skipping exon(s) 32, 34, (20 and 21), 24, 30, 41, 42, (5 and 6), (12 and 13), (26 and 27), (28 and 29), 35, 36, 19, or 43. More preferably, one or more antisense oligonucleotides are provided for skipping exon(s) (24, 30, 32, or 34), (30, 32 or 34), or 32 or 34. In some embodiments, exon 17, 32, 34, 35, 36, 41, 42, or a combination thereof, is skipped. In some embodiments, exon 24, 30, or a combination thereof, is skipped. In some embodiments, exon 32, 36 and 42, or a combination thereof, is skipped. In some embodiments, exon 32 and/or 36 is skipped. In some embodiments, only a single dysferlin exon is skipped. In some embodiments, the oligonucleotide comprises a sequence selected from SEQ ID NOS:1-19 and 21-34. In some embodiments, the sequence is selected from SEQ ID NOS:18 and 19. Preferably, the oligonucleotide comprises a sequence selected from SEQ ID NOS:1-54, more preferably, from SEQ ID NOS:19, 20, 6, 9, 12-15, 24, 25, 35, and 37. The oligonucleotides may be formulated into a composition, in particular, a pharmaceutical composition, for use in treating patients afflicted with a dysferlinopathy.

In one aspect, the disclosure provides nucleic acids comprising: a) an oligonucleotide or sets of oligonucleotides between 15 and 40 nucleotides and complementary to a dysferlin pre-mRNA to induce skipping exon(s) (2, 3, 4 and 5), (3 and 4), (5 and 6), 7, 8, 9, (10 and 11), (12 and 13), (14, 15 and 16), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52) or (53 and 54) or a combination thereof, and optionally b) a heterologous flanking sequence. In one aspect, the disclosure provides oligonucleotides or sets of oligonucleotides comprising between 15 and 40 nucleotides complementary to a dysferlin pre-mRNA to induce skipping exon(s) (2, 3, 4 and 5), (3 and 4), (5 and 6), 7, 8, 9, (10 and 11), (12 and 13), 14, (15, 16, 17 and 18), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52) or (53 and 54) or a combination thereof. Preferably, the oligonucleotides induce skipping exon(s) 32, 34, 36, 42, (20 and 21), (53 and 54), (31, 32 and 33), or a combination thereof. Preferably, oligonucleotides induce skipping exon(s) 32, 34, 36, 42, or (20 and 21). Preferably, the oligonucleotides induce skipping exon(s) 32, 34, (20 and 21), 24, 30, 41, 42, (5 and 6), (12 and 13), (26 and 27), (28 and 29), 35, 36, 19, or 43. More preferably, one or more antisense oligonucleotides are provided for skipping exon(s) (24, 30, 32, or 34), (30, 32 or 34), or 32 or 34. In some embodiments, exon 17, 32, 34, 35, 36, 41, 42, or a combination thereof, is skipped. In some embodiments, exon 24, 30, or a combination thereof, is skipped. In some embodiments, exon 32, 36 and 42, or a combination thereof, is skipped. In some embodiments, exon 32 and/or 36 is skipped. In some embodiments, only a single dysferlin exon is skipped. In some embodiments, the oligonucleotide comprises a sequence selected from SEQ ID NOS:1-19 and 21-34. In some embodiments, the sequence is selected from SEQ ID NOS:18 and 19. Preferably, the oligonucleotide comprises a sequence selected from SEQ ID NOS:1-54, more preferably from SEQ ID NOS:19, 20, 6, 9, 12-15, 24, 25, 35, and 37. In some embodiments, the heterologous flanking sequence is at least part of a nucleic acid delivery device. The nucleic acids may be formulated into a composition, in particular, a pharmaceutical composition, for use in treating patients afflicted with a dysferlinopathy.

The disclosure further provides a use of any of the oligonucleotides as disclosed herein for skipping a dysferlin exon. Preferably, an oligonucleotide hereof is used to skip a dysferlin exon in a cell having a mutation in the dysferlin gene.

The disclosure further provides methods for treating or alleviating symptoms associated with dysferlinopathies, comprising administering a therapeutic amount of a composition comprising one or more oligonucleotides of the invention.

A further aspect of the disclosure provides methods for skipping an exon in a pre-mRNA in a cell, the method comprising the improvement of providing a) a first antisense oligonucleotide capable of inducing skipping of the exon in a wild-type form of the pre-mRNA and b) a second antisense oligonucleotide capable of inducing skipping of the exon in a wild-type form of the pre-mRNA.

Preferably, a method is provided for skipping an exon in a pre-mRNA in a cell comprising selecting a first oligonucleotide that induces skipping of at least 5% of the exon as assessed by RT-PCR in cells expressing a wild-type faun of the pre-mRNA, further selecting a second oligonucleotide that induces skipping of at least 5% of the exon as assessed by RT-PCR in cells expressing a wild-type form of the pre-mRNA, and providing the cell with the first and second oligonucleotides.

In some embodiments, the oligonucleotides are independently capable (at a concentration of 500 nM or less) of inducing skipping of the exon in a wild-type form of the pre-mRNA at levels of at least 5% as assessed by RT-PCR in cells expressing the pre-mRNA. In some embodiments, the exon comprises a non-sense or missense mutation resulting in a protein with reduced function. In some embodiments, the first and second antisense oligonucleotides are complementary to non-overlapping regions of the wild-type form of the pre-mRNA. In some embodiments, the first and second antisense oligonucleotides are at least 80% complementary to the wild-type form of the pre-mRNA. In some embodiments, the first oligonucleotide and the best-aligned region of the wild-type form of the pre-mRNA have 8, 6, preferably 4, or, more preferably, 2 or fewer mismatches, and the second oligonucleotide and the best-aligned region of the wild-type form of the pre-mRNA have 8, 6, preferably 4, or, more preferably, 2 or fewer mismatches. In some embodiments, the oligonucleotides are provided to a cell having a pre-mRNA that comprises a mutation that reduces the complementarity of the first or second oligonucleotide to the pre-mRNA. In some embodiments, the mutation reduces the ability of the first or second oligonucleotide to induce exon-skipping. In some embodiments, the mutation that reduces complementarity is also the non-sense or missense mutation that results in a protein with reduced function. In some embodiments, one or both of the first and second oligonucleotides are complementary to the wild-type exon. In some embodiments, one or both of the first and second oligonucleotides are complementary to at least one predicted exonic splicing enhancer site or exon inclusion signal of the exon RNA. In some embodiments, one or both of the first and second oligonucleotides are complementary to a wild-type intron flanking the exon. In some embodiments, one or both of the first and second oligonucleotides are complementary to at least one predicted intronic splicing enhancer site of the wild-type intron. In some embodiments, the pre-mRNA does not encode dysregulin, clotting factor VIII or thyroglobulin. In some embodiments, the pre-mRNA encodes for a protein selected from dysferlin, collagen VI alpha 1, myotubular myopathy 1, laminin-alpha 2, and calpain 3. In some embodiments, the pre-mRNA comprises three or more exons.

In some embodiments, the disclosure provides the use of the oligonucleotides for decreasing the amount of an undesired protein, preferably an onco-gene or viral protein, in a cell. In some embodiments, a subject afflicted with a tumor, cancer, or viral infection is administered a pharmaceutical composition comprising the first and second oligonucleotide in an amount sufficient to induce exon skipping.

In some embodiments, the disclosure provides the use of the oligonucleotides for increasing the amount of functional protein in a cell by skipping an exon in a pre-mRNA comprising a mutation. In some embodiments, a subject having a mutated pre-mRNA, preferably harboring a missense or nonsense mutation, is administered a pharmaceutical composition comprising the first and second oligonucleotides in an amount sufficient to induce exon skipping.

In one aspect, the disclosure provides a set of two or more oligonucleotides, each independently capable of inducing skipping of an exon in a wild-type form of a pre-mRNA in a cell. The set of two or more oligonucleotides may be used in the methods disclosed herein and may be formulated in a pharmaceutical composition. The disclosure further provides a composition for skipping an exon in a pre-mRNA comprising two oligonucleotides, wherein the first oligonucleotide induces skipping of at least 5, preferably 10, more preferably 20, or more preferably 40% or more of the exon as assessed by RT-PCR in cells expressing a wild-type form of the pre-mRNA and the second oligonucleotide induces skipping of at least 5, preferably 10, more preferably 20, or more preferably 40% or more of the exon as assessed by RT-PCR in cells expressing a wild-type form of the pre-mRNA. As used herein to assess exon skipping, 5% exon skipping, for example, refers to the exon being skipped in 5% of the pre-mRNAs.

In one aspect, the disclosure provides methods for skipping an exon in a pre-mRNA in a cell, the improvement comprising selecting an oligonucleotide complementary to at least part of a 150 bp intron sequence flanking the exon, wherein at least part of the 150 bp intron sequence hybridizes to at least part of the exon; and providing the oligonucleotide to the cell. In some embodiments, the oligonucleotide is not complementary to a branch point, an acceptor splice site or a donor splice site. In some embodiments, hybridization of the oligonucleotide to the intron affects the secondary structure of the exon. In some embodiments, hybridization of the oligonucleotide to the intron disrupts the secondary structure of the exon. In some embodiments, the oligonucleotide is not complementary to an intron-splicing enhancer. In some embodiments, the pre-mRNA does not encode apolipoprotein B, cystic fibrosis transmembrane conductance regulator, or dysregulin. In some embodiments, the pre-mRNA encodes a protein selected from dysferlin, collagen VI alpha 1, myotubular myopathy 1, laminin-alpha 2, and calpain 3. In some embodiments, the oligonucleotide is complementary to the intron sequence downstream of the exon. In some embodiments, the pre-mRNA comprises three or more exons. In some embodiments, the exon is less than 500 bp. In some embodiments, the pre-mRNA is dysferlin pre-mRNA and the skipped-exon is selected from 2, 8, 9, 10, 14, 15, 17, 35. In some embodiments, the exon comprises a non-sense or missense mutation.

One aspect of the disclosure provides an oligonucleotide capable of inducing the skipping of an exon in a pre-mRNA, wherein the oligonucleotide is complementary to at least part of a 150 bp intron sequence flanking the exon and at least part of the 150 bp intron sequence hybridizes to at least part of the exon. The oligonucleotide may be used in the methods disclosed herein and may be formulated in a pharmaceutical composition.

One aspect of the disclosure provides a method of selecting an exon-skipping oligonucleotide, comprising: selecting a contiguous region of a pre-mRNA that comprises at least part of the exon to be skipped and at least part of an intronic sequence flanking the exon, determining the predicted secondary structure of the selected contiguous region, and designing an oligonucleotide sequence that is complementary to at least part of an intronic sequence predicted to hybridize to at least part of the exon, wherein the oligonucleotide is capable of inducing skipping of at least 5% of the exon as assessed by RT-PCR in cells expressing a wild-type form of the pre-mRNA. Compositions, preferably pharmaceutical compositions, comprising the selected oligonucleotides are also provided. Methods for skipping an exon in a pre-mRNA in a cell are further provided, wherein the method comprises selecting an oligonucleotide as described above and providing the oligonucleotide, or a pharmaceutical composition comprising the oligonucleotide, to the cell.

In some embodiments, the disclosure provides the use of the oligonucleotide for decreasing the amount of an undesired protein, preferably an onco-gene or viral protein, in a cell. In some embodiments, a subject afflicted with a tumor, cancer, or viral infection is administered a pharmaceutical composition comprising the oligonucleotide in an amount sufficient to induce exon skipping.

In some embodiments, the disclosure provides the use of the oligonucleotide for increasing the amount of functional protein in a cell by skipping an exon in a pre-mRNA comprising a mutation. In some embodiments, a subject having a mutated pre-mRNA, preferably harboring a missense or nonsense mutation, is administered a pharmaceutical composition comprising the oligonucleotide in an amount sufficient to induce exon skipping.

In one aspect, the disclosure provides a nucleic acid delivery vehicle comprising an oligonucleotide or sets of oligonucleotides comprising between 15-40 nucleotides that are complementary to a dysferlin pre-mRNA to induce skipping exon(s) (2, 3, 4 and 5), (3 and 4), (5 and 6), 7, 8, 9, (10 and 11), (12 and 13), (14, 15 and 16), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52), (53 and 54), or a combination thereof In one aspect, the disclosure provides a nucleic acid delivery vehicle comprising an oligonucleotide comprising between 15-40 nucleotides that are complementary to a dysferlin pre-mRNA to induce skipping exon(s) (2, 3, 4 and 5), (3 and 4), (5 and 6), 7, 8, 9, (10 and 11), (12 and 13), 14, (15, 16, 17 and 18), 17, (18, 19 and 20), (20 and 21), (22 and 23), 24, (26 and 27), (28 and 29), 30, (31, 32 and 33), 32, 34, 35, 36, 37, 38, (39 and 40), 41, 42, 43, (44, 45, 46 and 47), (46, 47 and 48), (50, 51, 52 and 53), (51 and 52) or (53 and 54) or a combination thereof. Preferably, the oligonucleotides induce skipping exon(s) 32, 34, 36, 42, (20 and 21), (53 and 54), (31, 32 and 33), or a combination thereof. Preferably, oligonucleotides induce skipping exon(s) 32, 34, 36, 42, or (20 and 21). Preferably, the oligonucleotides induce skipping exon(s) 32, 34, (20 and 21), 24, 30, 41, 42, (5 and 6), (12 and 13), (26 and 27), (28 and 29), 35, 36, 19, or 43. In some embodiments, exons (2, 3, 4 and 5), (3 and 4), (5 and 6), (10 and 11), (12 and 13), (14, 15 and 16), (18, 19 and 20), (20 and 21), (22 and 23), (26 and 27), (28 and 29), (31, 32 and 33), (39 and 40), (44, 45, 46 and 47), (46, 47 and 48), (50, 51, 52 and 53), (51 and 52), (53 and 54), or a combination thereof, are skipped. In some embodiments, exons (18, 19 and 20), (20 and 21), (22 and 23), (26 and 27), (28 and 29), (31, 32 and 33), (53 and 54), or a combination thereof, are skipped. In some embodiments, exons (18, 19 and 20), (20 and 21), (31, 32 and 33), (53 and 54), or a combination thereof, are skipped. In some embodiments, exons (18, 19 and 20), (20 and 21), (31, 32 and 33), or (53 and 54) are skipped. In some embodiments, exons (5 and 6), (12 and 13), (44, 45, 46 and 47), (50 and 51) or (52 and 53) are skipped. In some embodiments, the oligonucleotide is selected from SEQ ID NOS:1-19, and 21-34. Preferably, the oligonucleotide comprises a sequence selected from SEQ ID NOS:1-54, more preferably, from SEQ ID NOS:19, 20, 6, 9, 12-15, 24, 25, 35, and 37. In some embodiments, the nucleic acid delivery vehicle comprises an adeno-associated virus. In some embodiments, the disclosure provides the use of the nucleic acid delivery vehicle for the preparation of a medicament or pharmaceutical composition, in particular, a medicament for treating a dysferlinopathy.

In one aspect, the disclosure provides a pharmaceutical composition comprising one or more oligonucleotides as disclosed herein. In some embodiments, the composition further comprises a pharmaceutically acceptable carrier, filler, preservative, adjuvant, solubilizer, diluent and/or excipient.

### BRIEF DESCRIPTION OF THE DRAWINGS

FIG. 1. Dysferlin domains relative to DYSF exons. Dysferlin contains six or seven calcium-dependent C2 lipid-binding domains (C2), a transmembrane domain (T), a ferl domain (L), FerA and FerB domains (A and B, respectively) and Dysf_N and Dysf_C domains (N and C, respectively). The C2 and transmembrane domains have a function in membrane repair. The function of other domains is yet unknown.

FIG. 2. Antisense-mediated exon-skipping. Left panel: In this example, a mutation within exon 32 results in a premature stop codon (indicated by the transition of black to white in the pre-mRNA (top) and mRNA (middle), which leads to a prematurely truncated protein (bottom). Right panel: when antisense oligonucleotides (AON) targeting exon 32 are used, they will hybridize to this exon, thus hiding it from the splicing machinery, resulting in the skipping of this exon. Since exon 32 is in-frame (its length is divisible by 3), skipping will not disrupt the reading frame (the mRNA becomes black in the middle panel) and a full-length protein lacking the amino acids encoded by exon 32 will be generated (bottom).

FIG. 3. Dysferlin exons. In-frame exons are depicted in white, out-of-frame exons in black. Exons or combinations of exons can be skipped without disrupting the reading frame when the resulting ends fit (e.g., exons 39 and 40 can be skipped, since the end of exon 38 fits to the beginning of exon 41). 3a) An initial prediction of the reading frame of dysferlin having an error beginning at exon 15. 3b) A corrected version of the dysferlin exon structure. The predicted exons and combinations of exons that can be skipped does not change in the corrected version, with the exception that now exon 14 and the combination of (15, 16, 17, 18) is predicted in place of the previously predicted combination of (14, 15, 16).

FIG. 4. RT-PCR analysis of oligonucleotide-treated control cell cultures. h19DYSF2, h24DYSF1, h24DYSF2, h30DYSF1, h30DYSF2 and h34DYSF1 are effective, while h19DYSF1 and C (a control AON targeting the DMD (dystrophin) gene) are not. Correct exon-skipping was confirmed by sequence analysis (data not shown). No exon 19, 24, 32 or 34 skipping could be observed in non-treated (NT) cells, while for exon 30, low levels of physiological skipping were observed. Oligonucleotide treatment significantly increased these levels from <10% to >90%. No 32 exon skipping was observed with an oligonucleotide that targets exon 34 (h34DYSF2b). No 34 exon skipping was observed with an oligonucleotide that targets exon 32 (h32DYSF1b). Skipping percentages (assessed with Agilent Lab on a Chip) are indicated below each skip. Note that the intensity of the skip products is lower, due to the smaller fragment length (our efficiency assessment corrects for this). —RT and H₂O are negative controls. M is size marker.

FIG. 5. Properties of dysferlin exons.

FIG. 6. Summary of exon-skipping efficiency.

### DETAILED DESCRIPTION OF THE DISCLOSED EMBODIMENTS

Limb-Girdle Muscular Dystrophy type 2B (LGMD2B), Myoshi Myopathy (MM) and distal myopathy with anterior tibial onset (DMAT) are autosomal recessive allelic muscle diseases caused by mutations in the dysferlin-encoding DYSF gene, leading to severely reduced or complete absence of the dysferlin protein (Liu et al., 1998; Bashir et al., 1998, A gene related to Caenorhabditis elegans spermatogenesis factor fer-1 is mutated in limb-girdle muscular dystrophy type 2B, Nat. Genet. 20:37-42; Illa et al., 2001, Distal anterior compartment myopathy: a dysferlin mutation causing a new muscular dystrophy phenotype, Ann. Neurol. 49:130-134). Most dysferlinopathy patients have small mutations; stop- or frame-shift mutations lead to prematurely truncated proteins, while missense mutations generally affect protein stability (Therrien et al., 2006, Mutation impact on dysferlin inferred from database analysis and computer-based structural predictions, J. Neurol. Sci. 250:71-78). Over 100 different mutations have been reported in the Leiden Open Variation Database for almost 200 patients (see the world-wide web at dmd.nl).

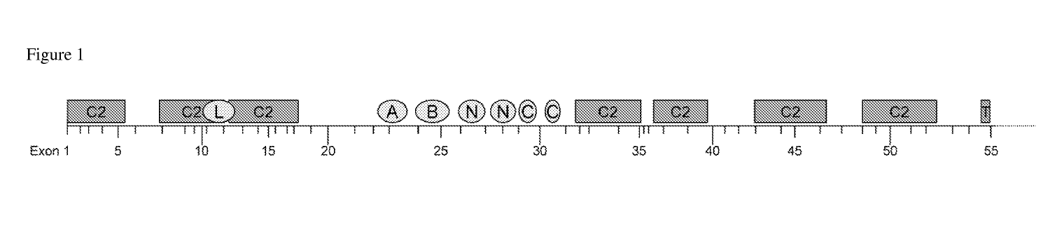

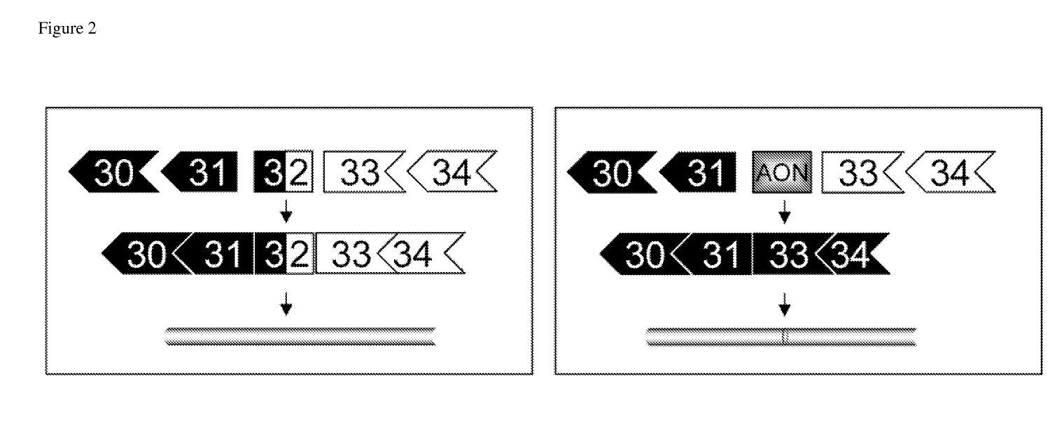

The dysferlin protein is expressed in many tissues, but most abundantly in heart and skeletal muscle (Bansal and Campbell, 2004). In the latter, the protein is located at the plasma membrane and in cytoplasmic vesicles (Bansal et al., 2003). It is thought that dysferlin has a function in vesicle trafficking and membrane patch fusion repair in muscle cells (Bansal and Campbell, 2004). Loss of dysferlin compromises skeletal muscle membrane repair and leads to progressive loss of muscle fibers (Bansal et al., 2003). The protein has several different domains (FIG. 1). The ENSEMBL database predicts six or seven calcium-dependent C2 lipid-binding (C2) domains, a transmembrane domain and multiple “fer” and “dysf” domains. The C2 domains probably mediate calcium-dependent vesicle fusion with the plasma membrane, while the transmembrane domains anchor the protein to the plasma membrane (Bansal and Campbell, 2004). The fer and dysf domains have, as yet, an unknown function (Therrien et al., 2006).

It is likely that parts of the dysferlin protein are redundant. The first indication for this is a finding by Sinnreich and colleagues that the mother of two LGMD2B patients was a compound heterozygote rather than a carrier (Sinnreich et al., 2006, Lariat branch point mutation in the dysferlin gene with mild limb-girdle muscular dystrophy, Neurology 66:1114-1116). One of the alleles contained a mutated branch point in intron 31, leading to skipping of exon 32. As exon 32 skipping does not disrupt the open reading frame, this resulted in a slightly shorter but apparently partly functional dysferlin protein at levels that were 10% of wild-type levels. The patient had only very mild proximal muscle weakness, elevated serum creatine kinase levels and was still ambulant at age 70. By contrast, her severely affected daughters were homozygous for a null mutation and had no dysferlin protein. In addition, a mildly affected patient has been presented with a dysferlin containing only the final two C2 and the transmembrane domains (Krahn et al., 2008, Partial functionality of a Mini-dysferlin molecule identified in a patient affected with moderately severe primary dysferlinopathy, Neuromuscul. Disord. 18:781). This patient was ambulant without a cane at age 41. Further proof for the functionality of this protein came from its proper location at the sarcolemma and the delivery of a gene encoding this “minidysferlin” into a dysferlin-negative mouse model through an adeno-associated viral vector. This resulted in detectable levels of the mini-dysferlin protein and an improved phenotype.

Thus, bypassing dysferlin mutations may lead to more stable and/or more functional dysferlin proteins and would, therefore, have therapeutic potential. A way to achieve this is the modulation of dysferlin pre-mRNA splicing using antisense oligonucleotides (AONs) or antisense sequences, which hide target exons from the splicing machinery, such that they are not included into the final mRNA (“exon-skipping”) (FIG. 2).

Exon-skipping is a technique used for restructuring mRNA that is produced from pre-mRNA exhibiting undesired splicing in a subject. The restructuring may be used to decrease the amount of protein produced by the cell. Exon-skipping interferes with the natural splicing processes occurring within a eukaryotic cell. In higher eukaryotes, the genetic information for proteins in the DNA of the cell is encoded in exons that are separated from each other by intronic sequences. These introns are in some cases very long. The transcription machinery of eukaryotes generates a pre-mRNA, which contains both exons and introns, while the splicing machinery, often already during the production of the pre-mRNA, generates the actual coding region for the protein by splicing together the exons present in the pre-mRNA.

Exon-skipping results in mature mRNA that lacks at least one skipped exon. Thus, when the exon codes for amino acids, exon-skipping leads to the expression of an altered product. Technology for exon-skipping is currently directed towards the use of antisense oligonucleotides (AONs).

Promising results with exon-skipping have recently been reported with a therapy aimed at restoring the reading frame of the dystrophin pre-mRNA in cells from Duchenne's Muscular Dystrophy (DMD) patients. See, e.g., PCT Publication Nos. WO2006/000057, WO02/024906, WO2004/083446, WO2006/112705, WO2007/135105, and WO2009/054725, which are hereby incorporated by reference in their entirety. In both DMD and Becker muscular dystrophy (BMD), the muscle protein dystrophin is affected. In DMD, dystrophin is absent, whereas in BMD dystrophin is present but at reduced levels and/or abnormally formed. By the targeted skipping of a specific exon, a DMD is converted into a milder BMD phenotype, thereby partially rescuing activity.

In many genes, deletion of an entire exon leads to the production of a non-functional protein through the loss of important functional domains or the disruption of the reading frame. The present disclosure is based, in part, on the surprising finding that exon-skipping can be efficiently used to affect the splicing of dysferlin mRNA and restore at least partial function of a DYSF mutation. Accordingly, the disclosure provides compositions and methods for providing a cell with an alternatively spliced dysferlin mRNA. The compositions and methods are useful for increasing the ratio of wild-type to mutant DYSF protein in a cell and thus may be used in the treatment of dysferlin-related muscular dystrophies, e.g., limb girdle muscular dystrophy 2B and Miyoshi myopathy.

As used herein, “to comprise” and its conjugations is used in its non-limiting sense to mean that items following the word are included, but items not specifically mentioned are not excluded. In addition, the verb “to consist” may be replaced by “to consist essentially of,” meaning that a compound or adjunct compound as defined herein may comprise additional component(s) than the ones specifically identified, the additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition), whereas, if it is administered after manifestation of the unwanted condition, the treatment is therapeutic (i.e., it is intended to diminish, ameliorate, or stabilize the existing unwanted condition or side effects thereof).

The disclosure provides a selection of DYSF exons as suitable targets for antisense-mediated exon-skipping. In some embodiments, the selection is based on the protein encoding domains and reported mutations. The disclosure also provides guidelines for identifying oligonucleotides that can be used to induce exon-skipping (see Example 1).

One aspect of the disclosure provides methods and compositions for exon-skipping a DYSF exon. In some embodiments, exon-skipping results in the generation of a dysferlin encoding mRNA wherein:
exon 1 is spliced to exon 6 (2, 3, 4 and 5 skip);
exon 2 is spliced to exon 5 (3 and 4 skip);
exon 4 is spliced to exon 7 (5 and 6 skip);
exon 6 is spliced to exon 8 (7 skip);
exon 7 is spliced to exon 9 (8 skip);
exon 8 is spliced to exon 10 (9 skip);
exon 9 is spliced to exon 12 (10 and 11 skip);
exon 11 is spliced to exon 14 (12 and 13 skip);
exon 13 is spliced to exon 15 (exon 14 skip)
exon 14 is spliced to exon 19 (exon 15, 16, 17 and 18 skip)
exon 16 is spliced to exon 18 (17 skip);
exon 17 is spliced to exon 21 (18, 19 and 20 skip);
exon 19 is spliced to exon 22 (20 and 21 skip);
exon 21 is spliced to exon 24 (22 and 23 skip);
exon 23 is spliced to exon 25 (24 skip);
exon 25 is spliced to exon 28 (26 and 27 skip);
exon 27 is spliced to exon 30 (28 and 29 skip);
exon 29 is spliced to exon 31 (30 skip);
exon 30 is spliced to exon 34 (31, 32 and 33 skip);
exon 31 is spliced to exon 33 (32 skip);
exon 33 is spliced to exon 35 (34 skip);
exon 34 is spliced to exon 36 (35 skip);
exon 35 is spliced to exon 37 (36 skip);
exon 36 is spliced to exon 38 (37 skip);
exon 37 is spliced to exon 39 (38 skip);
exon 38 is spliced to exon 41 (39 and 40 skip);
exon 40 is spliced to exon 42 (41 skip);
exon 41 is spliced to exon 43 (42 skip);
exon 42 is spliced to exon 44 (43 skip);
exon 43 is spliced to exon 48 (44, 45, 46 and 47 skip);
exon 45 is spliced to exon 49 (46, 47 and 48 skip);
exon 49 is spliced to exon 54 (50, 51 52 and 53 skip);
exon 50 is spliced to exon 53 (51 and 52 skip); or
exon 52 is spliced to exon 55 (53 and 54 skip).

In some embodiments, exon 7, 8, 9, 17, 24, 30, 32, 34, 35, 36, 37, 38, 41, 42 or 43 of dysferlin, or a combination thereof, is skipped. In some embodiments, exon 17, 32, 34, 35, 36, 41, 42, or a combination thereof, is skipped. In some embodiments, exon 24, 30, or a combination thereof, is skipped. In some embodiments, exon 32, 36 and 42, or a combination thereof, is skipped. Preferably, exon 32 and/or 36 is skipped. In some embodiments, only a single dysferlin exon is skipped. More preferably, one or more antisense oligonucleotides are provided for skipping exon(s) 24, 30, 32, or 34; 30, 32 or 34; or 32 or 34.

One aspect of the disclosure provides methods and compositions for skipping more than one exon in a dysferlin pre-mRNA. This embodiment, referred to as double- or multi-exon-skipping (see, e.g., A. Aartsma-Rus, et al., Am. J. Hum. Genet. 2004, 74(1):83-92; and A. Aartsma-Rus, et al., Exploring the frontiers of therapeutic exon-skipping for Duchenne muscular dystrophy by double targeting within one or multiple exons, Mol. Ther. 2006, 14(3):401-7). Multi-exon skipping refers to the skipping of more than one exon resulting in a shortened, but at least partly functional protein. Preferably, multi-exon skipping targets a single mutation. For example, in compound heterozygotes, or rather, individuals having a different mutation on each allele, it is preferred that only one of the mutant alleles is targeting for exon-skipping. The skipping may result in the deletion of one or more exons; however, it is not the intention to provide one oligonucleotide for the mutation on the first allele and a second oligonucleotide for the mutation on the second allele.

In some embodiments, an oligonucleotide that is capable of inhibiting inclusion of a dysferlin exon into dysferlin mRNA is combined with at least one other oligonucleotide capable of inhibiting inclusion of another dysferlin exon into dysferlin mRNA. In some embodiments, an oligonucleotide is used that is complementary to a first exon of a dysferlin pre-mRNA and wherein an oligonucleotide is used that is complementary to a second exon of dysferlin pre-mRNA. This way, inclusion of two or more exons of a dysferlin pre-mRNA in mRNA produced from this pre-mRNA is prevented. In most cases, double-exon-skipping results in the exclusion of only the two targeted exons from the dysferlin pre-mRNA. However, in other cases, the targeted exons and the entire region in between the exons, including intervening exons, in the pre-mRNA are not present. Combinations of exons to be skipped include adjacent exons that together are in-frame (i.e., the total number of nucleotides is divisible by 3), such as the exon combinations (20 and 21), (53 and 54), (22 and 23), (26 and 27), (28 and 29), (18, 19 and 20), and (31, 32 and 33). A skilled person is aware that any of the oligonucleotides described herein are useful in exon-skipping and may be used together with another oligonucleotide described herein to induce multiple exon skipping or may be used with oligonucleotides described elsewhere for exon skipping.

In some embodiments, exons (2, 3, 4 and 5), (3 and 4), (5 and 6), (10 and 11), (12 and 13), (14, 15 and 16), (18, 19 and 20), (20 and 21), (22 and 23), (26 and 27), (28 and 29), (31, 32 and 33), (39 and 40), (44, 45, 46 and 47), (46, 47 and 48), (50, 51, 52 and 53), (51 and 52), (53 and 54), or a combination thereof, are skipped. In some embodiments, exons (2, 3, 4 and 5), (3 and 4), (5 and 6), (10 and 11), (12 and 13), (15, 16, 17 and 18), (18, 19 and 20), (20 and 21), (22 and 23), (26 and 27), (28 and 29), (31, 32 and 33), (39 and 40), (44, 45, 46 and 47), (46, 47 and 48), (50, 51, 52 and 53), (51 and 52), (53 and 54), or a combination thereof, are skipped.

In some embodiments, exons (18, 19 and 20), (20 and 21), (22 and 23), (26 and 27), (28 and 29), (31, 32 and 33), (53 and 54), or a combination thereof, are skipped.

In some embodiments, exons (18, 19 and 20), (20 and 21), (31, 32 and 33), (53 and 54), or a combination thereof, are skipped. In some embodiments, exons (18, 19 and 20), (20 and 21), (31, 32 and 33), or (53 and 54) are skipped.

In some embodiments, exons (5 and 6), (12 and 13), (44, 45, 46 and 47), (50 and 51) or (52 and 53) are skipped.

In some embodiments of the methods and compositions described herein, two or more oligonucleotides selected from SEQ ID NOS:1-54 are provided.

In some embodiments, two oligonucleotides are provided that are complementary to a first and second dysferlin exon that are separated in dysferlin pre-mRNA by at least one exon. It is also possible to specifically promote the skipping of the intervening exons by providing a linkage between the two complementary oligonucleotides. Hence, in one embodiment, oligonucleotides complementary to at least two dysferlin exons are separated by a linking moiety. The oligonucleotides are thus linked in this embodiment so as to form a single molecule. The linkage may be through any means, but is preferably accomplished through a nucleotide linkage. In the latter case, the number of nucleotides that do not contain an overlap between one or the other complementary exon can be zero, but is preferably between 4 to 40 nucleotides. The linking moiety can be any type of moiety capable of linking oligonucleotides. Preferably, the linking moiety comprises at least four uracil nucleotides.

The skipping of an exon is induced by the binding of one or more oligonucleotides targeting pre-mRNA. Splicing of a pre-mRNA occurs via two sequential transesterification reactions. First, the 2′OH of a specific branch-point nucleotide within the intron that is defined during spliceosome assembly performs a nucleophilic attack on the first nucleotide of the intron at the 5′ splice site forming the lariat intermediate. Second, the 3′OH of the released 5′ exon then performs a nucleophilic attack at the last nucleotide of the intron at the 3′ splice site, thus joining the exons and releasing the intron lariat. The branch point and splice sites of an intron are thus involved in a splicing event. In some embodiments, an oligonucleotide comprising a sequence that is complementary to such branch point and/or splice site is used for exon-skipping.

Since splice sites contain consensus sequences, the use of an oligonucleotide comprising a sequence that is complementary to a splice site involves the risk of promiscuous hybridization. Hybridization of oligonucleotides to splice sites other than the sites of the exon to be skipped could easily interfere with the accuracy of the splicing process. To overcome these and other potential problems related to the use of oligonucleotides that are complementary to a branch point and/or splice site sequence, one embodiment disclosed herein provides methods and compositions wherein an oligonucleotide comprises a sequence that is complementary to a dysferin pre-mRNA exon. In some embodiments, the oligonucleotide is capable of specifically inhibiting an exon inclusion signal of at least one exon in the dysferin pre-mRNA. Interfering with an exon inclusion signal (EIS) has the advantage that such elements are located within the exon. By providing an oligonucleotide for the interior of the exon to be skipped, it is possible to interfere with the exon inclusion signal, thereby effectively masking the exon from the splicing apparatus. The failure of the splicing apparatus to recognize the exon to be skipped thus leads to exclusion of the exon from the final mRNA. This embodiment does not interfere directly with the enzymatic process of the splicing machinery (the joining of the exons). It is thought that this allows the method to be more specific and/or reliable. It is thought that an EIS is a particular structure of an exon that enables the splicing machinery to recognize the exon. However, the disclosure is certainly not limited to this model. It has been found that agents capable of binding to an exon are capable of inhibiting an EIS. An oligonucleotide may specifically contact the exon at any point and still be able to specifically inhibit the EIS.

In some embodiments, an oligonucleotide directed toward an exon internal sequence typically exhibits no overlap with non-exon sequences. In some embodiments, the oligonucleotide does not overlap with the splice sites, at least not insofar as these are present in the intron. In some embodiments, an oligonucleotide directed toward an exon internal sequence preferably does not contain a sequence complementary to an adjacent intron.

In some embodiments, an oligonucleotide comprises a sequence that is complementary to a region of a dysferlin pre-mRNA exon that is hybridized to another part of a dysferlin pre-mRNA exon (closed structure), and a sequence that is complementary to a region of a dysferlin pre-mRNA exon that is not hybridized to another part of the dysferlin pre-mRNA (open structure). Without being bound by theory, it is thought that the overlap with an open structure improves the invasion efficiency of the oligonucleotide (i.e., increases the efficiency with which the oligonucleotide can enter the structure), whereas the overlap with the closed structure subsequently increases the efficiency of interfering with the secondary structure of the RNA of the exon, and thereby interferes with the exon inclusion signal. (See PCT Publication No. WO 2004/083432.)

The disclosure further provides methods and compositions wherein an exon-skipping oligonucleotide is complementary to a binding site for a serine-arginine (SR) protein in RNA of an exon of a dysferlin pre-mRNA. In PCT publication WO 2006/112705, we have disclosed the presence of a correlation between the effectivity of an exon-internal antisense oligonucleotide (AON) in inducing exon-skipping and the presence of a (for example, by ESEfinder) predicted SR binding site in the target pre-mRNA site of the oligonucleotide. Therefore, in one embodiment, an oligonucleotide is generated comprising determining a (putative) binding site for an SR (Ser-Arg) protein in RNA of a dysferlin exon and producing an oligonucleotide that is complementary to the RNA and that at least partly overlaps the (putative) binding site. The term “at least partly overlaps” is defined herein as to comprise an overlap of only a single nucleotide of an SR binding site as well as multiple nucleotides of the binding site as well as a complete overlap of the binding site. This embodiment may further comprise determining from a secondary structure of the RNA, a region that is hybridized to another part of the RNA (closed structure) and a region that is not hybridized in the structure (open structure), and subsequently generating an oligonucleotide that at least partly overlaps the (putative) binding site and that overlaps at least part of the closed structure and overlaps at least part of the open structure. In this way, we increase the chance of obtaining an oligonucleotide that is capable of interfering with the exon inclusion from the pre-mRNA into mRNA. It is possible that a first selected SR-binding region does not have the requested open-closed structure, in which case, another (second) SR protein binding site is selected that is then subsequently tested for the presence of an open-closed structure. This process is continued until a sequence is identified that contains an SR protein binding site as well as a(n) (partly overlapping) open-closed structure. This sequence is then used to design an oligonucleotide that is complementary to the sequence.

Such a method for generating an oligonucleotide is also performed by reversing the described order, i.e., first generating an oligonucleotide comprising determining, from a secondary structure of RNA from a dysferlin exon, a region that hybridizes to another part of the RNA (closed structure) and a region that is not hybridized in the structure (open structure), and subsequently generating an oligonucleotide, of which at least a part of the oligonucleotide is complementary to the closed structure and of which at least another part of the oligonucleotide is complementary to the open structure. This is then followed by determining whether an SR protein binding site at least overlaps with the open/closed structure. In this way, the method of WO 2004/083432 is improved. In yet another embodiment, the selections are performed simultaneously.

Without wishing to be bound by theory, it is currently thought that use of an oligonucleotide directed to an SR protein binding site results in (at least partly) impairing the binding of an SR protein to the binding site of an SR protein, which results in disrupted or impaired splicing.

Preferably, an open/closed structure and an SR protein binding site partly overlap and even more preferred, an open/closed structure completely overlaps an SR protein binding site or an SR protein binding site completely overlaps an open/closed structure. This allows for an improved disruption of exon inclusion.

The disclosure further provides methods and compositions wherein an exon-skipping oligonucleotide is capable of specifically binding a regulatory RNA sequence, which is required for the correct splicing of a dystrophin exon in a transcript. Several cis-acting RNA sequences are involved in the correct splicing of exons in a transcript. In particular, supplementary elements, such as intronic or exonic splicing enhancers (ISEs and ESEs) or silencers (ISSs and ESEs), are identified to regulate specific and efficient splicing of constitutive and alternative exons. Using sequence-specific antisense oligonucleotides (AONs) that bind to the elements, their regulatory function is disturbed so that the exon is skipped. Hence, in one embodiment, an oligonucleotide is used that is complementary to an intronic splicing enhancer (ISE), an exonic splicing enhancer (ESE), an intronic splicing silencer (ISS) and/or an exonic splicing silencer (ESS).

One aspect of the disclosure provides oligonucleotide sequences useful for the methods and compositions for dysferlin exon-skipping as described herein. In some embodiments, an oligonucleotide is selected from one or more of the following sequences:

| (SEQ ID NO: 1) | |
|---|---|
| h17DYSF1 | GCU UGA CAG CAC CUG CAG GC |
| (SEQ ID NO: 2) | |
| h17DYSF2 | AGG CUU UCG AAG GCU UGA CA |
| (SEQ ID NO: 3) | |
| h18DYSF1 | CAU AGA GGU UGA UGU AGC AG |
| (SEQ ID NO: 4) | |
| h18DYSF2 | GGU CUG GGA AGC CUG UGA AC |
| (SEQ ID NO: 5) | |
| h19DYSF1 | GAA GCC GGC CAC GAU AAG CC |
| (SEQ ID NO: 6) | |
| h19DYSF2 | CCU UCU GUU CAC UGU GCU CC |
| (SEQ ID NO: 7) | |
| h20DYSF1 | UGG CAU CAU CCA CAU CCU GC |
| (SEQ ID NO: 8) | |
| h20DYSF2 | GGU CAU GUC GAA CUU GUU CC |
| (SEQ ID NO: 9) | |
| h20DYSF3 | GGC AGG UCA UGU CGA ACU UG |
| (SEQ ID NO: 10) | |
| h21DYSF1 | ACC ACC ACA GGU UUC ACG |
| (SEQ ID NO: 11) | |
| h21DYSF2 | GCA GCU GGU UCU GAG UCU CG |
| (SEQ ID NO: 12) | |
| h24DYSF1 | GCA UCC AGA UGA CGA UGU CCG |
| (SEQ ID NO: 13) | |
| h24DYSF2 | GCU UCC CAC AAU UCU UGC CA |
| (SEQ ID NO: 14) | |
| h30DYSF1 | CCG UCU UCU CCA GUG GCU CC |
| (SEQ ID NO: 15) | |
| h30DYSF2 | CGG CGG AAG GCA UCU GUC UUG |
| (SEQ ID NO: 16) | |
| h31DYSF1 | UGG AAU CUU CAC UCU UGU CA |
| (SEQ ID NO: 17) | |
| h31DYSF2 | UCG UGG GUC UGU UCA CAC CG |
| (SEQ ID NO: 18) | |
| h32DYSF2 | GCG UAG AUG GUA GCG GU |
| (SEQ ID NO: 19) | |
| h32DYSF3 | GAG UCC UUG UCC AUC GCA GC |
| (SEQ ID NO: 20) | |
| h32DYSF1* | UCC GUU CCA GAC UCG GUU CAC |
| (h34DYSF2b) | |
| (SEQ ID NO: 21) | |
| h33DYSF1 | GUG UUC UUC ACC ACC ACC GU |
| (SEQ ID NO: 22) | |
| h33DYSF2 | GGC GGU UGC UCA GCA ACU G |
| (SEQ ID NO: 23) | |
| h33DYSF3 | CUU CAC CAC CAC CGU CUU CUG |
| (SEQ ID NO: 24) | |
| h34DYSF1 | CGA CGG CUG GCU GCC CCU CGU C |
| (SEQ ID NO: 25) | |
| h34DYSF2* | GCA GCG UAG AUG GUA GCG GU |
| (h32DYSF1b) | |
| (SEQ ID NO: 26) | |
| h35DYSF1 | CAA AAC CAG GAA UAU GGU GG |
| (SEQ ID NO: 27) | |
| h36DYSF1 | CAU CCA GGA UCC UUG AUG UC |
| (SEQ ID NO: 28) | |
| h42DYSF1 | GGC CUC CAC AUU CUC CAG CU |
| (SEQ ID NO: 29) | |
| h42DYSF2 | UGU CUC CUC CUG CGU CUU GC |
| (SEQ ID NO: 30) | |
| h42DYSF3 | UGG AUC UUC UGU CUC CUC CU |
| (SEQ ID NO: 31) | |
| h54DYSF1 | AAC UUC AUG GUC UUG UAU GG |
| (SEQ ID NO: 32) | |
| h54DYSF2 | GAU GAA GAU GGC CAG GAA CA |
| (SEQ ID NO: 33) | |
| h53DYSF1 | CUG CUA CAA UCU CCA AGG UC |
| (SEQ ID NO: 34) | |
| h53DYSF2 | AGG CCG CUC CUC AUG CUC A |
| (SEQ ID NO: 35) | |
| h43DYSF1 | GGC CUC CAC AUU CUC CAG CU |
| (SEQ ID NO: 36) | |
| h43DYSF2 | UGU CUC CUC CUG CGU CUU GC |
| (SEQ ID NO: 37) | |
| h43DYSF3 | UGG AUC UUC UGU CUC CUC CU |
| (SEQ ID NO: 38) | |
| h20DYSF4 | UAC UUG CGC CUC CUA AGG UAC |
| (SEQ ID NO: 39) | |
| h20DYSF5 | UAC UUG CGC CUC CUA AGG UA |
| (SEQ ID NO: 40) | |
| h20DYSF6 | UUG CGC CUC CUA AGG UAC U |
| (SEQ ID NO: 41) | |
| h20DYSF7 | AUG GUG GCU GAG UAG AAG G |
| (SEQ ID NO: 42) | |
| h20DYSF8 | GAU GGC AUC AUC CAC AUC CU |
| (SEQ ID NO: 43) | |
| h20DYSF9 | AUG CUG ACC UCA AAC UGG AU |
| (SEQ ID NO: 44) | |
| h20DYSF10 | UCG AAC UUG UUC CCG UAG UU |
| (SEQ ID NO: 45) | |
| h20DYSF11 | UCG AAC UUG UUC CCG UAG UUC |
| (SEQ ID NO: 46) | |
| h20DYSF12 | UCA UGU CGA ACU UGU UCC CGU |
| (SEQ ID NO: 47) | |
| h21DYSF3 | GGU AGG UAG UAG UAG UGG CA |
| (SEQ ID NO: 48) | |
| h21DYSF4 | ACA GGU UUC ACG UUA CCC CA |
| (SEQ ID NO: 49) | |
| h21DYSF5 | UGA UGU CCU CCC AGU AGG A |
| (SEQ ID NO: 50) | |
| h21DYSF6 | GAU UCU AUG GCU GAU GUC CUC |
| (SEQ ID NO: 51) | |
| h21DYSF7 | CUC GAU UCU AUG GCU GAU GUC |
| (SEQ ID NO: 52) | |
| h21DYSF8 | CUG GUU CUG AGU CUC GAU UC |
| (SEQ ID NO: 53) | |
| h21DYSF9 | AGC AAU CCC AAG CAG CUG GUU |
| (SEQ ID NO: 54) | |
| h21DYSF10 | CAC CAC AGG UUU CAC GUU AC |

* After reviewing the sequences, it was realized that h32DYSF2 (SEQ ID NO: 20) and h34DYSF2 (SEQ ID NO: 25) had been inadvertently swapped. SEQ ID NO: 20 is now designated as h34DYSF2b to indicate that it targets exon 34 and SEQ ID NO: 25 is now designated as h32DYSF1b to indicate that it targets exon 32.

An oligonucleotide is also selected from the reverse complement of any one of SEQ ID NOs: 1-54. The nomenclature “h17DYSF1” refers to a first oligonucleotide directed to targeting the 17^{th} exon of the human DYSF gene. A skilled person appreciates that while the sequences list “U”, referring to a uracil base, the sequences also encompass the use of thymine in place of the uracil. The particular nomenclature was used for consistency with the RNA-based oligonucleotides described in the Examples.

Methods and compositions are provided that comprise one or more oligonucleotides having between 15 and 40 nucleotides and comprising a sequence selected from SEQ ID NOs:1-34, preferably 1-54, more preferably from 19, 20, 6, 9, 12-15, 24, 25, 35, and 37. In some embodiments, one or more oligonucleotides is selected from SEQ ID NOs: 1-19 and 21-34. In some embodiments, the oligonucleotides are at least 80, 85, 90, 98, 98, or 100% identical to SEQ ID NOs:1-19 and 21-34 and are capable of inducing exon-skipping in dysferlin pre-mRNA. In some embodiments, the oligonucleotides have a sequence that differs by less than 10, 8, 6, preferably 4, or more preferably by 2 amino acids from SEQ ID NOs:1-19 and 21-34. In some embodiments, the oligonucleotides are at least 80, 85, 90, 98, 98, or 100% identical to SEQ ID NOs:1-54 and are capable of inducing exon-skipping in dysferlin pre-mRNA. In some embodiments, the oligonucleotides have a sequence that differs by less than 10, 8, 6, preferably 4, or more preferably by 2 amino acids from SEQ ID NOs:1-54. In some embodiments, oligonucleotides comprise additional heterologous flanking sequences, e.g., vector/plasmid sequences or additional sequences to modify stability or binding characteristics.

The oligonucleotide(s) for skipping one or more dysferlin exons may be formulated in a pharmaceutical composition useful, e.g., for the administration to subjects afflicted with a dysferlinopathy.

A further aspect of the disclosure provides methods and compositions for exon-skipping, wherein an oligonucleotide is provided that comprises a sequence complementary to a non-exon region of a pre-mRNA. In some embodiments, an oligonucleotide complementary to at least part of the intron sequence flanking the exon is provided, wherein at least part of the intron sequence hybridizes to at least part of the exon. In some embodiments, an oligonucleotide is complementary to at least part of a 500, 400, 300, 200, preferably 150, more preferably 100, or more preferably 50 by intron sequence flanking the exon and at least part of the 500, 400, 300, 200, 150, 100, or 50 by intron sequence, respectively, hybridizes to at least part of the exon. In some embodiments, the oligonucleotide is complementary to at least 5, 10, 15, 20, 25, 30, 35, 40, 45 or more contiguous intronic nucleotides. In some embodiments, the oligonucleotide is complementary to at least part of an intron sequence that is downstream of the exon.

RNA molecules exhibit strong secondary structures, mostly due to base pairing of complementary or partly complementary stretches within the same RNA. It has long since been thought that structures in the RNA play a role in the function of the RNA. Without being bound by theory, it is believed that the secondary structure of the RNA of an exon plays a role in structuring the splicing process. This secondary structure may be due to interactions within an exon or between an exon and neighboring intron sequences. Through its structure, an exon is recognized as a part that needs to be included in the mRNA. Herein, this signaling function is referred to as an exon inclusion signal. A complementary oligonucleotide of this embodiment is capable of interfering with the structure of the exon and thereby capable of interfering with the exon inclusion signal of the exon. In some embodiments, hybridization of the oligonucleotide to at least part of the intron affects, and in some embodiments disrupts, the secondary structure of the exon. In some embodiments, hybridization of the oligonucleotide masks the exon from the splicing machinery.

The secondary structure is best analyzed in the context of the pre-mRNA wherein the exon resides. Such structure may be analyzed in the actual RNA. However, it is currently possible to predict the secondary structure of an RNA molecule (at lowest energy costs) quite well using structure-modeling programs. A non-limiting example of a suitable program is RNA mfold version 3.1 server (L. Cartegni, et al., Nat. Rev. Genet. 2002, 3(4):285-98). A person skilled in the art will be able to predict, with suitable reproducibility, a likely structure of the exon, given the nucleotide sequence. Best predictions are obtained when providing such modeling programs with both the exon and flanking intron sequences. It is typically not necessary to model the structure of the entire pre-mRNA. In some embodiments, a nucleic acid stretch of less than 5000 bp, 3000 bp, 2000 bp, 1000 bp, 500 bp, 800 bp, 600 bp, or 400 bp of sequence is used to model the secondary structure. In some embodiments, the nucleic acid stretch comprises less than 500 bp, 200 bp, or preferably 150 bp of intronic sequence flanking an exon.

Targeting of intronic sequences may be particularly useful when the exon to be skipped is relatively small. In some embodiments, the skipped exon is less than 2000, 1000, 500, 400, 300, 200, 100, or 50 bp. In some embodiments, the skipped exon is exon 2, 8, 9, 10, 14, 15, 17, or 35 of dysferlin.

Exon-skipping using oligonucleotides directed to 5′ and 3′ splice sites, as well as branch points, has been described. In some embodiments of the methods and compositions of the present disclosure, oligonucleotides complementary to intronic sequences are not complementary to a 5′ splice site, a 3′ splice site, or a branch point. Furthermore, in some embodiments, the oligonucleotide is not complementary to an intron splicing enhancer. In some embodiments, the pre-mRNA does not encode for a gene selected from apolipoprotein B, cystic fibrosis transmembrane conductance regulator, dystrophin, or dysregulin.

In some embodiments of the methods, the oligonucleotides are provided to a cell having at least one mutation in the pre-mRNA of their target that reduces the stability, expression, and/or function of the corresponding mRNA or protein. Preferably the mutation is a non-sense or missense mutation in the exon to be skipped. Addition of the exon-skipping oligonucleotide results in the production of an mRNA and/or protein with increased stability, expression, and/or function as compared to the mutated form.

In some embodiments, the methods and compositions using intron complementary oligonucleotides provide single-exon skipping. This is particularly useful when the pre-mRNA comprises a non-sense or missense mutation in the targeted exon.

The disclosure provides oligonucleotides useful for the above-described methods and pharmaceutical compositions comprising the oligonucleotides. The pharmaceutical compositions are useful for administering to a subject having a mutation in a gene selected from Duchenne muscular dystrophy gene, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), and/or a calpain 3 gene (CAPN3).

The use of two oligonucleotides for exon-skipping has been previously described in methods for targeting more than one exon (PCT publication WO 2006/000057) and for improving exon-skipping efficiency for exons having, e.g., multiple independent exon splice enhancer sites (PCT publication WO 2007/135105). The present disclosure is directed to providing methods and compositions having at least two oligonucleotides, wherein each oligonucleotide can effectively induce exon-skipping of the same exon. The oligonucleotides may be provided in nucleic acid vehicles, such as vectors.

Skipping of a particular exon or particular exons can result in a restructured mRNA that encodes a shorter than normal but at least partially functional protein. A practical problem in the development of a medicament based on exon-skipping technology is the plurality of mutations that may result in a deficiency of a functional protein in the cell. Despite the fact that different mutations can be corrected by the skipping of a single exon, this plurality of mutations requires the generation of a large number of different pharmaceuticals. Patients having mutations that affect oligonucleotide hybridization, and thus exon-skipping, would need to be treated with an alternative pharmaceutical having a different oligonucleotide. Furthermore, polymorphisms, such as single nucleotide polymorphisms (SNPs), are present in a number of genes and these sequence variants may have reduced complementarity to an exon-skipping oligonucleotide. As used herein, a polymorphism refers to a sequence variant having a frequency of at least 1% in the population, while a mutation refers to a variant having a frequency of less than 1%.

An advantage of a composition comprising at least two oligonucleotides that target a single exon and effectively induce exon-skipping is that the composition can be administered to a larger number of patients, including those who carry a mutation that would affect the exon-skipping of one of the oligonucleotides. This property is very useful in that only a limited number of pharmaceuticals need to be generated for treating many different mutations in a gene.

One aspect of the disclosure, therefore, provides methods and compositions that comprise at least two oligonucleotides that target exon-skipping of the same exon in a pre-mRNA, wherein each oligonucleotide is capable of inducing skipping of the exon in a wild-type form of the pre-mRNA. In some embodiments, each oligonucleotide is capable of inducing skipping in a wild-type form of the pre-mRNA at levels of at least 5% as assessed by RT-PCR. A person skilled in the art will appreciate that other assays can be used to assess exon-skipping and are discussed in further detail herein.

The oligonucleotides are generally at least 80, 90, 95, 99, or 100% complementary to wild-type pre-mRNA. In some embodiments, the first oligonucleotide and the best-aligned region of the wild-type form of the pre-mRNA have 8, 6, preferably 4, or more preferably 2 or fewer mismatches. In some embodiments, the second oligonucleotide and the best-aligned region of the wild-type form of the pre-mRNA have 8, 6, preferably 4, or more preferably 2 or fewer mismatches.

In preferred embodiments, the oligonucleotides are complementary to non-overlapping regions of the wild-type pre-mRNA.

In some embodiments of the methods, the oligonucleotides are provided to a cell having at least one mutation in the pre-mRNA of their target that reduces the stability, expression, and/or function of the corresponding mRNA or protein. Preferably, the mutation is a non-sense or missense mutation in the exon to be skipped. Addition of the exon-skipping oligonucleotide results in the production of an mRNA and/or protein with increased stability, expression, and/or function as compared to the mutated form.

In some embodiments, the pre-mRNA comprises at least one mutation or polymorphism that reduces the complementarity of at least one of the oligonucleotides to the pre-mRNA and thereby, optionally, reducing the induction of exon-skipping. The mutation or polymorphism in the pre-mRNA may lead to the reduction of the stability, expression, and/or function of the corresponding mRNA or protein or it may have no or little affect on the mRNA or protein.

The oligonucleotides useful for the methods and compositions may be directed to exon and/or intron sequences as described herein, including, but not limited to, exonic splicing enhancer sites, exon inclusion signals, and intron splicing enhancer sites.

The disclosure provides oligonucleotides useful for the above-described methods and pharmaceutical compositions comprising the oligonucleotides. The pharmaceutical compositions are useful for administering to a subject having a mutation in a gene selected from, e.g., Duchenne muscular dystrophy gene, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), and/or a calpain 3 gene (CAPN3). In some embodiments, the pre-mRNA is dysferlin mRNA. In some embodiments, the pre-mRNA does not encode dysregulin, dystrophin, clotting factor VIII, or thyroglobulin.

In some embodiments, the first and second oligonucleotides are formulated in a single composition, which is, preferably, suitable for administration to a human subject. In some embodiments, the first and second oligonucleotide are linked together as a single molecule as described previously herein. While not wishing to be bound by theory, it is believed that by providing a second oligonucleotide that can efficiently induce exon-skipping of the same exon, a single pharmaceutical composition can be administered on a significantly greater patient population. Preferably, the pharmaceutical composition is formulated to comprise therapeutically effective amounts of the individual oligonucleotides.

For instance, production of an undesired protein can be at least in part reduced by inhibiting inclusion of a required exon into the mRNA. A preferred method of the invention further comprises allowing translation of mRNA produced from splicing of the pre-mRNA. Preferably, the mRNA encodes a functional protein. In a preferred embodiment, the protein comprises two or more domains, wherein at least one of the domains is encoded by the mRNA as a result of skipping at least part of an exon in the pre-mRNA.

In some embodiments, a method or composition as described herein is used to at least in part decrease the production of an aberrant protein. Such proteins can, for instance, be onco-proteins or viral proteins. In many tumors, not only the presence of an onco-protein but also its relative level of expression, has been associated with the phenotype of the tumor cell. Similarly, not only the presence of viral proteins but also the amount of viral protein in a cell determines the virulence of a particular virus. Moreover, for efficient multiplication and spread of a virus, the timing of expression in the life cycle and the balance in the amount of certain viral proteins in a cell determines whether viruses are efficiently or inefficiently produced. Using a method of the invention, it is possible to lower the amount of aberrant protein in a cell such that, for instance, a tumor cell becomes less tumorigenic (metastatic) and/or a virus-infected cell produces less virus.

The methods and compositions for inducing exon-skipping disclosed herein utilize oligonucleotides, in particular, antisense oligonucleotides, which target pre-mRNA. As used herein, antisense oligonucleotides (AONs) are single strands of DNA or RNA that are complementary to a target sequence. Methods for designing exon-skipping oligonucleotides have been described herein, as well as in the art (see, e.g., Aartsma-Rus et al., 2005, Functional analysis of 114 exon-internal AONs for targeted DMD exon-skipping: indication for steric hindrance of SR protein binding sites. Oligonucleotides 15:284-297; Aartsma-Rus et al., 2008, Guidelines for Antisense AON Design and Insight Into Splice-modulating Mechanisms, Mol. Ther. 2009 Mar. 17(3):548-53, Epub 2008; and PCT Publication Nos. WO2006/000057 and WO2007/135105).

The oligonucleotide and the pre-mRNA are complementary to each other when a sufficient number of corresponding positions in each molecule are occupied by nucleotides that can hydrogen bond with each other. Thus, “specifically hybridizable” and “complementary” are terms that are used to indicate a sufficient degree of complementarity or precise pairing, such that stable and specific binding occurs between the oligonucleotide and the RNA target. It is understood in the art that the sequence of an antisense molecule need not be 100% complementary to that of its target sequence to be specifically hybridizable.

The term “complementarity” is used herein to refer to a stretch of nucleic acids, i.e., contiguous nucleic acids, that can hybridize to another stretch of nucleic acids under physiological conditions. It is thus not absolutely required that all the bases in the region of complementarity are capable of pairing with bases in the opposing strand. For instance, when designing the oligonucleotide, one may want to incorporate, for instance, a residue that does not base pair with the base on the complementary strand. Mismatches may to some extent be allowed, if under the circumstances in the cell, the stretch of nucleotides is capable of hybridizing to the complementary part.

In some embodiments, a complementary part comprises at least 3, 4, 5, 10, 15, 20, 25, 30, 35, 40, or more consecutive nucleotides. The complementary regions are preferably designed such that, when combined, they are specific for the target pre-mRNA. Such specificity may be created with various lengths of complementary regions as this depends on the actual sequences in other (pre-)mRNA in the system. The risk that also one or more other pre-mRNA will be able to hybridize to the oligonucleotide decreases with increasing size of the oligonucleotide. It is clear that oligonucleotides comprising mismatches in the region of complementarity but that retain the capacity to hybridize to the targeted region(s) in the pre-mRNA, can be used in the present invention.

It is thought that higher hybridization strengths (i.e., increasing number of interactions with the opposing strand) are favorable in increasing the efficiency of the process of interfering with the splicing machinery of the system. In some embodiments, the complementarity is between 90 and 100%. In general, this allows for approximately one or two mismatch(es) in an oligonucleotide of around 20 nucleotides

In some embodiments, an oligonucleotide of the methods and compositions described herein comprises a sequence that is complementary to part of a target pre-mRNA, such that the complementary part is at least 50% of the length of the oligonucleotide, more preferably at least 60%, even more preferably at least 70%, even more preferably at least 80%, even more preferably at least 90% or even more preferably at least 95%, or even more preferably 98% or more.

The length of an oligonucleotide useful in the methods and compositions described herein may vary so long as it is capable of binding selectively to the intended location within the pre-mRNA molecule. The length of such sequences can be determined in accordance with selection procedures described herein. Generally, the oligonucleotide will be from about 10 nucleotides in length up to about 50 nucleotides in length. It will be appreciated, however, that any length of nucleotides within this range may be used in the method. In some embodiments, the length of the complementary part of the oligonucleotide is at least 13, 14, 15, 16, 17, 18, 19, 20, 21, 22, 23, 24, 25, 26, 27, 28, 29, 30 nucleotides. In some embodiments, an oligonucleotide is complementary to a consecutive part of between 13 and 50, between 16 and 50, between 15 and 40, between 15 and 25, or between 20-25 nucleotides of pre-mRNA.

In a preferred embodiment, an oligonucleotide is complementary to between 15 and 40 nucleotides of pre-mRNA and has less than 10, 8, 6, or, preferably, 4 mismatches with the pre-mRNA.

Complementarity can be expressed by the number of mismatches between an oligonucleotide and its base pairing to the target region of the pre-mRNA. The target region refers to the contiguous pre-mRNA sequences, or the complement thereof, that best aligns with the oligonucleotide. In such a comparison, if gaps exist, it is preferable that such gaps are counted as mismatches.

Amino acid and polynucleotide alignments, percentage sequence identity, and degree of complementarity may be determined for purposes using the ClustalW algorithm using standard settings: see the world wide web at ebi.ac.uk/emboss/align/index.html, Method: EMBOSS::water (local): Gap Open=10.0, Gap extend=0.5, using Blosum 62 (protein), or DNAfull for nucleotide/nucleobase sequences.

As will be understood, depending on context, “mismatch” refers to a nonidentity in sequence (as, for example, between the nucleobase sequence of an oligonucleotide and the reverse complement of the target region to which it binds) or to noncomplementarity in sequence (as, for example, between an oligonucleotide and the target region to which it binds).

An oligonucleotide used in the methods and compositions described herein may comprise flanking sequences, i.e., heterologous flanking sequences, in addition to a sequence that is complementary to part of a target pre-mRNA. Additional flanking sequences may be used to modify the binding of a protein to the oligonucleotide, to modify a thermodynamic property of the oligonucleotide, or to modify target RNA binding affinity. Additional flanking sequences may also be part of a nucleic acid delivery vehicle, such as a vector or plasmids, e.g., an adeno-associated virus.

Different types of nucleic acid may be used to generate the oligonucleotides useful in the methods and compositions described herein. The term “oligonucleotide,” as used herein, refers to a polynucleoside formed from a plurality of linked nucleoside units. Such oligonucleotides can be obtained from existing nucleic acid sources, including genomic or cDNA, as well as production by synthetic methods. In some embodiments, each nucleoside unit includes a heterocyclic base and a pentofuranosyl, trehalose, arabinose, 2′-deoxy-2′-substituted arabinose, 2′-O-substituted arabinose or hexose sugar group. The nucleoside residues can be coupled to each other by any of the numerous known internucleoside linkages. Such internucleoside linkages include, without limitation, phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkage (e.g., (Rp)- or (Sp)-phosphorothioate, alkylphosphonate, or phosphotriester linkages). In some embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides, and oligonucleotides having backbone sections with alkyl linkers or amino linkers.

The oligonucleotides provided in the disclosure can include naturally occurring nucleosides, modified nucleosides, or mixtures thereof. The term “nucleoside” as used herein, refers to a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, and non-standard nucleosides; see, e.g., PCT Publication Nos. WO 92/07065 and WO 93/15187). There are several examples of modified nucleic acid bases known in the art as summarized by Limbach et al., 1994, Nucleic Acids Res. 22:2183. Some of the non-limiting examples of chemically modified and other natural nucleic acid bases that can be introduced into nucleic acids include: inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, quesosine, 2-thiouridine, 4-thiouridine, wybutosine, wybutoxosine, 4-acetylcytidine, 5 -(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35:14090). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule. In some embodiments, the modified nucleoside is a 2′-substituted ribonucleoside, an arabinonucleoside or a 2′-deoxy-2′-substituted-arabinoside.

In some embodiments, the oligonucleotide does not consist of DNA. In exemplary embodiments, the oligonucleotide comprises RNA, as RNA/RNA hybrids are very stable. Since one of the aims of the exon-skipping technique is to direct splicing in subjects, it is preferred that the oligonucleotide RNA comprises a modification providing the RNA with an additional property, for instance, resistance to endonucleases and RNaseH, additional hybridization strength, increased stability (for instance, in a bodily fluid), increased or decreased flexibility, reduced toxicity, increased intracellular transport, tissue-specificity, etc. An exemplary modification comprises a 2′-O-methyl-phosphorothioate oligoribonucleotide modification, in particular, a 2′-O-methyl-phosphorothioate oligodeoxyribonucleotide modification. The disclosure thus provides methods and compositions, wherein an oligonucleotide is used that comprises RNA that contains a modification, such as a 2′-O-methyl modified ribose (RNA) or deoxyribose (DNA) modification.

In some embodiments, oligonucleotides comprise LNAs in which the 2′-hydroxyl group is linked to the 4′ carbon atom of the sugar ring, thereby forming a 2′-C,4′-C-oxymethylene linkage, thereby forming a bicyclic sugar moiety. Potent and nontoxic antisense oligonucleotides containing LNAs have been described (Wahlestedt et al., Proc. Natl. Acad. Sci. U.S.A., 2000, 97:5633-5638). LNA displays high target affinity and low toxicity and, therefore, induces a high efficiency of exon-skipping.

In some embodiments, oligonucleotides comprise peptide nucleic acids PNAs, as described in A. N. Elayadi and D. R. Corey, Curr. Opin. Investig. Drugs 2001, 2(4):558-61; and H. J. Larsen, et al., Biochim. Biophys. Acta. 1999, 1489(1):159-66. In some embodiments, oligonucleotides comprise morpholino phosphorodiamidate. (J. Summerton and D. Weller, Antisense Nucleic Acid Drug Dev. 1997, 7(3):187-95.)

Hybrid oligonucleotides are also suitable for the methods and compositions disclosed herein. A “hybrid oligonucleotide” is an oligonucleotide having more than one type of nucleoside. One example of such a hybrid oligonucleotide comprises a ribonucleotide or 2′-substituted ribonucleotide region, and a deoxyribonucleotide region (see, e.g., Metelev and Agrawal, U.S. Pat. Nos. 5,652,355, 6,346,614 and 6,143,881). In some embodiments, the disclosure provides a hybrid oligonucleotide comprising an oligonucleotide comprising a 2′-O-methyl-phosphorothioate oligo(deoxy)ribonucleotide modification and locked nucleic acid. This particular combination comprises better sequence specificity compared to an equivalent consisting of locked nucleic acid, and comprises improved effectivity when compared with an oligonucleotide consisting of 2′-O-methyl-phosphorothioate oligo(deoxy)ribonucleotide modification.

An exon-skipping technique as described herein is preferably applied such that the absence of one or more exons from mRNA produced from a pre-mRNA generates a coding region for a functional—albeit shorter than wild-type—protein. In this context, inhibiting inclusion of one or more exons may be measured by the detection of the original mRNA, which is decreased by at least about 10% as assessed by RT-PCR or that a corresponding protein is decreased of at least about 10% as assessed by immunofluorescence or Western blot analysis. The decrease is preferably of at least 20%, 30%, 40%, 50%, 60%, 70%, 80%, 90% or 100% in a cell.

Inhibiting inclusion of one or more exons may also be measured by the detection of the shorter exon-skipped mRNA or exon-skipped protein product. In some embodiments, the oligonucleotides of the methods and compositions described herein induce skipping of an exon at least 5, 10, 20, 30, 40, 50, 60, 70, 80, or 90% as assessed by RT-PCR in cells expressing the pre-mRNA. In some embodiments, the oligonucleotides induce skipping at a concentration of 0.1 M, 0.01 M, 0.001 M, 500 nM, 100, nM, or less.

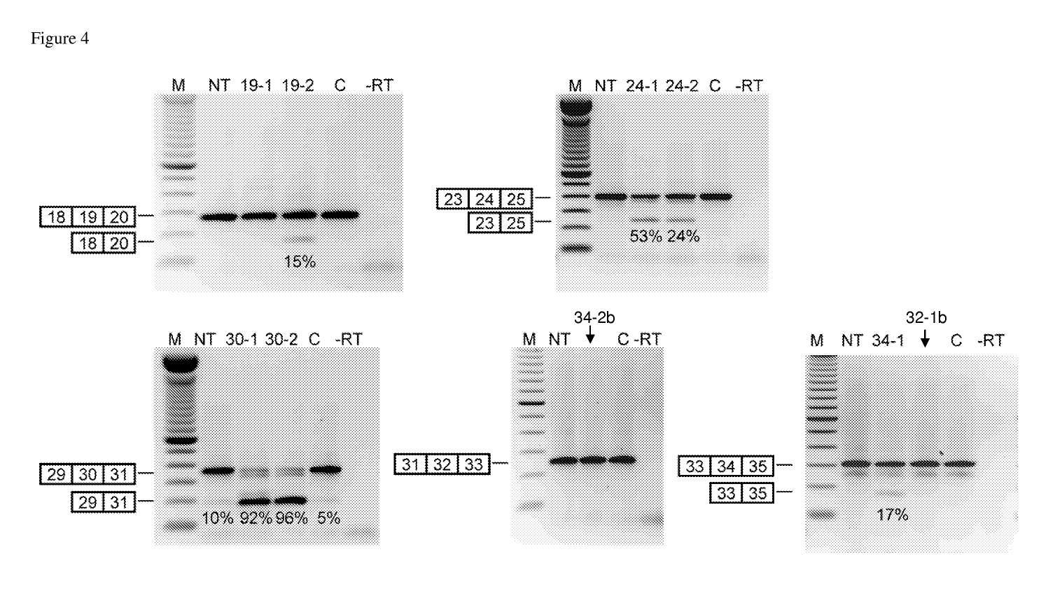

Gene expression level is preferably assessed using classical molecular biology techniques such as (real time) PCR, specific qPCR, TaqMan analysis, lab-on-a-chip analysis of RT-PCR fragments, densitometry analysis of RT-PCR fragments, PCR using radioactive or fluorescent isotopes, arrays, or Northern analysis. A steady-state level of a protein is determined directly by quantifying the amount of a protein. Quantifying a protein amount may be carried out by any known technique such as Western blotting or immunoassay using an antibody raised against a protein. The skilled person will understand that alternatively or in combination with the quantification of a gene expression level and/or a corresponding protein, the quantification of a substrate of a corresponding protein or of any compound known to be associated with a function or activity of a corresponding protein or the quantification of the function or activity of a corresponding protein using a specific assay may be used to assess the alteration of an activity or steady-state level of a protein. See, e.g., Example 2 and FIG. 4.

The different conformation of the mRNA/protein results from the skipping of one or more exons. However, when potential (cryptic) splice acceptor and/or donor sequences are present within the targeted exon, occasionally a new exon inclusion signal is generated defining a different (neo) exon, i.e., with a different 5′ end, a different 3′ end, or both. This type of activity is within the scope of the present invention as the targeted exon is excluded from the mRNA. The presence of a new exon, containing part of the targeted exon, in the mRNA does not alter the fact that the targeted exon, as such, is excluded. The inclusion of a neo-exon can be seen as a side effect, which occurs only occasionally. There are two possibilities when exon-skipping is used to restore (part of) an open reading frame of a gene that is disrupted as a result of a mutation. One is that the neo-exon is functional in the restoration of the reading frame, whereas in the other case, the reading frame is not restored. When selecting oligonucleotides for restoring reading frames by means of exon-skipping, it is, of course, clear that under these conditions, only those oligonucleotides are selected that indeed result in exon-skipping that restores the open reading frame, with or without a neo-exon.

The methods and compositions disclosed herein are useful for restructuring mRNA that is produced from pre-mRNA exhibiting undesired splicing in a subject. The restructuring may be used to decrease the amount of protein produced by the cell. This is useful when the cell produces a particular undesired protein, e.g., an onco-gene or a viral protein.

In some embodiments, however, restructuring of the mRNA via exon-skipping is used to promote the production of a functional protein in a cell, i.e., restructuring leads to the generation of a coding region for a functional protein. The latter embodiment is preferably used to restore an open reading frame that was lost as a result of a mutation. Exemplary genes comprise a Duchenne muscular dystrophy gene, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), and/or a calpain 3 gene (CAPN3); however, a skilled person will appreciate that the disclosed methods and compositions may be used to alter the splicing of a variety of genes.

In some embodiments, the methods and compositions disclosed herein target dysferlin and promote the production of functional dysferlin protein. Alleviating one or more symptom(s) of dysferlinopathy in an individual may be assessed by any of the following assays: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, and improvement of the quality of life. Each of these assays is known to the skilled person. For each of these assays, as soon as a detectable improvement or prolongation of a parameter measured in an assay has been found, it will preferably mean that one or more symptoms of a dysferlinopathy has been alleviated in an individual using a method or composition disclosed herein. Alternatively, the alleviation of one or more symptom(s) of a dysferlinopathy may be assessed by measuring an improvement of muscle fiber function, integrity and/or survival.

The improvement of muscle fiber function, integrity and/or survival may be assessed using at least one of the following assays: a detectable decrease of creatine kinase in blood, a detectable increase of calcium-dependent membrane repair response, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in S. Hodgetts et al, (2006), Neuromuscular Disorders, 16:591-602. A detectable decrease in creatine kinase may mean a decrease of 5%, 10%, 20%, 30%, 40%, 50%, 60%, 70%, 80%, 90% or more compared to the concentration of creatine kinase in the same individual before treatment.

A detectable decrease of necrosis of muscle fibers may be assessed in a muscle biopsy, such as, e.g., using biopsy cross-sections (S. Hodgetts et al. (2006)). A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in the same individual before treatment.

A detectable increase of the homogeneity of the diameter of a muscle fiber may be assessed in a muscle biopsy cross-section, see, e.g., S. Hodgetts et al. (2006).

A detectable increase of calcium-dependent membrane repair response may be assessed as described in, e.g., D. Bansal et al., Nature (2003) 423:168-172; and N. J. Lennon, J. Biol. Chem. (2003) 278:50466-50473. A detectable increase of calcium-dependent membrane repair may mean an increase of 5%, 10%, 20%, 30%, 40%, 50%, 60%, 70%, 80%, 90% or more compared to the membrane repair in the muscle fibers from the same individual before treatment.

A treatment according to the methods disclosed herein is about at least one week, about at least one month, about at least several months, about at least one year, about at least 2, 3, 4, 5, 6 years or more.

Oligonucleotides useful in the methods and compositions described herein may be suitable for direct administration to a cell, tissue and/or an organ in vivo of individuals and may be administered directly in vivo, ex vivo or in vitro. Alternatively, suitable means for providing cells with an oligonucleotide are present in the art. An oligonucleotide may, for example, be provided to a cell in the form of an expression vector, wherein the expression vector encodes a transcript comprising the oligonucleotide. The expression vector is preferably introduced into the cell via a gene delivery vehicle.

A preferred delivery vehicle is a viral vector such as an adeno-associated virus vector (AAV), or a retroviral vector such as a lentivirus vector and the like (A. Goyenvalle et al., Science 2004, 306(5702):1796-9). Plasmids, artificial chromosomes, plasmids suitable for targeted homologous recombination and integration in the human genome of cells may also be suitably applied for delivery of an oligonucleotide as defined herein. Preferred for the current invention are those vectors wherein transcription is driven from PolIII promoters, and/or wherein transcripts are in the form fusions with U1 or U7 transcripts, which yield good results for delivering small transcripts. It is within the skill of the artisan to design suitable transcripts, such as PolIII driven transcripts or in the form of a fusion transcript with a U1 or U7 transcript (M. A. Denti et al., Hum. Gene Ther. 2006, 17(5):565-74; and L. Gorman et al., Proc. Natl. Acad. Sci. U. S. A. 1998, 95(9):4929-34).

Oligonucleotides will usually be administered to a mammal as a pharmaceutical composition that includes the oligonucleotide and any pharmaceutically acceptable suitable adjuvants, carriers, excipients, and/or stabilizers, and can be in solid or liquid form, such as tablets, capsules, powders, solutions, suspensions, or emulsions. Nucleic acids can be administered in an effective carrier, e.g., any formulation or composition capable of effectively delivering the nucleic acid to cells in vivo. Nucleic acids contained within viral vectors can be delivered to cells in vivo by infection or transduction using virus. Nucleic acids and vectors can also be delivered to cells by physical means, e.g., by electroporation, lipids, cationic lipids, liposomes, DNA gun, calcium phosphate precipitation, injection, or delivery of naked nucleic acid.

Methods for delivering nucleic acid compounds are known in the art (see, e.g., Akhtar et al., 1992, Trends Cell Bio. 2:139; and Delivery Strategies for Antisense Oligonucleotide Therapeutics, ed. Akhtar, 1995; Sullivan et al., PCT Publication No. WO 94/02595). These protocols can be utilized for the delivery of virtually any nucleic acid compound. Nucleic acid compounds can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to, oral (tablet or pill form) and/or intrathecal delivery (Gold, 1997, Neuroscience 76:1153-1158). Other approaches include the use of various transport and carrier systems, for example, through the use of conjugates and biodegradable polymers. For a comprehensive review on drug delivery strategies, see Ho et al., 1999, Curr. Opin. Mol. Ther. 1:336-343; and Jain, Drug Delivery Systems: Technologies and Commercial Opportunities, Decision Resources, 1998; and Groothuis et al., 1997, J. Neuro. Virol. 3:387-400. More detailed descriptions of nucleic acid delivery and administration are provided in Sullivan et al., supra, Draper et al., PCT WO93/23569, Beigelman et al., PCT Publication No. WO99/05094, and Klimuk et al., PCT Publication No. WO99/04819.

When administering the oligonucleotide to an individual, it is preferred that the oligonucleotide is dissolved in a solution that is compatible with the delivery method. For intravenous, subcutaneous, intramuscular, intrathecal and/or intraventricular administration, it is preferred that the solution is a physiological salt solution.

Excipients may also be used that are capable of forming complexes, vesicles and/or liposomes with oligonucleotides. Many of these excipients are known in the art. Suitable excipients comprise polyethylenimine (PEI), or similar cationic polymers, including polypropyleneimine or polyethylenimine copolymers (PECs) and derivatives, ExGen 500, synthetic amphiphils (SAINT-18), LIPOFECTIN™, DOTAP and/or viral capsid proteins. Such excipients have been shown to efficiently deliver nucleic acids to a wide variety of cultured cells, including muscle cells. Their high transfection potential is combined with an excepted low to moderate toxicity in terms of overall cell survival.

The amount to be administered will, of course, vary depending upon the treatment regimen. Generally, the pharmaceutical composition is administered to achieve an amount effective for amelioration of, or prevention of the development of symptoms of, the hemorrhagic condition (i.e., a therapeutically effective amount). Thus, a therapeutically effective amount can be an amount that is capable of at least partially preventing or reversing the hemorrhagic condition. The dose required to obtain an effective amount may vary depending on the agent, formulation, disease or disorder, and individual to whom the agent is administered.

Determination of effective amounts may also involve in vitro assays in which varying doses of agent are administered to cells in culture and the concentration of agent effective for ameliorating some or all symptoms is determined in order to calculate the concentration required in vivo. Effective amounts may also be based on in vivo animal studies. A therapeutically effective amount can be determined empirically by those of skill in the art.

In some embodiments, a concentration of an oligonucleotide as defined herein, which is ranged between about 0.1 nM and about 1 nM, between about 0.3 nM to about 400 nM, or between about 1 nM to about 200 nM is used. If several oligonucleotides are used, this concentration may refer to the total concentration of oligonucleotides or the concentration of each oligonucleotide added. The ranges of concentration of oligonucleotide(s) as given above are preferred concentrations for in vitro or ex vivo uses. The skilled person will understand that depending on the oligonucleotide(s) used, the target cell to be treated, the gene target and its expression levels, the medium used and the transfection and incubation conditions, the concentration of oligonucleotide(s) used may further vary and may need to be further optimized.

An oligonucleotide used in the methods and compositions described herein is synthetically produced and administered directly to a cell, a tissue, an organ and/or patients in formulated form in a pharmaceutically acceptable composition or preparation. The delivery of a pharmaceutical composition to the subject is preferably carried out by one or more parenteral injections, e.g., intravenous and/or subcutaneous and/or intramuscular and/or intrathecal and/or intraventricular administrations, preferably injections, at one or at multiple sites in the human body.

All patent and literature references cited in the present specification are hereby incorporated by reference in their entirety.

The invention is further explained in the following examples. These examples do not limit the scope of the invention, but merely serve to clarify the invention.

### EXAMPLES

### General Methods

### Database

Mutations as reported on May 8, 2009 in the DYSF LOVD database (available on the world-wide web at dmd.nl) were analyzed.

### Oligonucleotides

Oligonucleotide design was based on our guidelines for DMD exons and focused on targeting partially open secondary RNA structures (predicated by m-fold (Zuker, 2003)), the presence of predicted RESCUE-ESE and SC35 and the absence of predicted Tra2β sites (using the human splicing finder (Desmet et al., 2009, Human Splicing Finder: an online bioinformatics tool to predict splicing signals, Nucleic Acids Res. 37:e67) and favorable binding energy (Aartsma-Rus et al., 2008; Zuker, 2003). All oligonucleotides (SEQ ID NOS:5, 6, 12, 13, 15, 16, 20, 24, 25) target exon-internal sequences and consist of 2′-O-methyl RNA with a full-length phosphorothioate backbone and were manufactured by Eurogentec (Belgium).

### Cell Culture and Transfection

Human control myoblasts were cultured and differentiated as described previously (Aartsma-Rus et al., 2003). Oligonucleotides were transfected at a 500 nM concentration using 2.5 μl polyethyleneimine (MBI-Fermentas) per μg oligonucleotide, following manufacturer's instructions. An unrelated five-fluorescein-labeled oligonucleotide targeting exon 45 of the dystrophin gene was used to confirm the efficiency of transfection (>90%).

### RNA Analysis

RNA was isolated >28 hours after transfection using RNA-Bee (Campro Scientific) according to the manufacturer's instruction. An RT-PCR was performed with random hexamer primers, as described (Aartsma-Rus et al., 2004). Primers flanking the targeted exons (sequence on request) were used to amplify the cDNA as described previously for dystrophin (Aartsma-Rus et al., 2003, Therapeutic antisense-induced exon-skipping in cultured muscle cells from six different DMD patients, Hum. Mol. Genet. 12:907-14), but using a single PCR for 35 cycles. Skip products were analyzed by sequencing analysis as described (Aartsma-Rus et al., 2003).

### Example 1

### Guidelines for Targeting DYSF Exon-Skipping

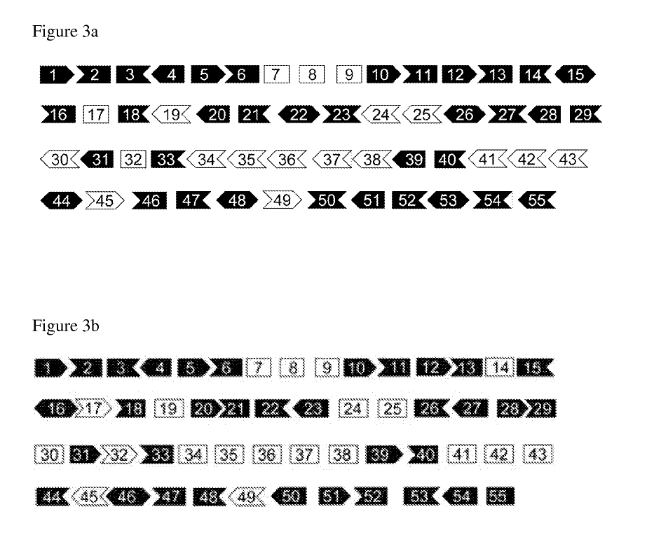

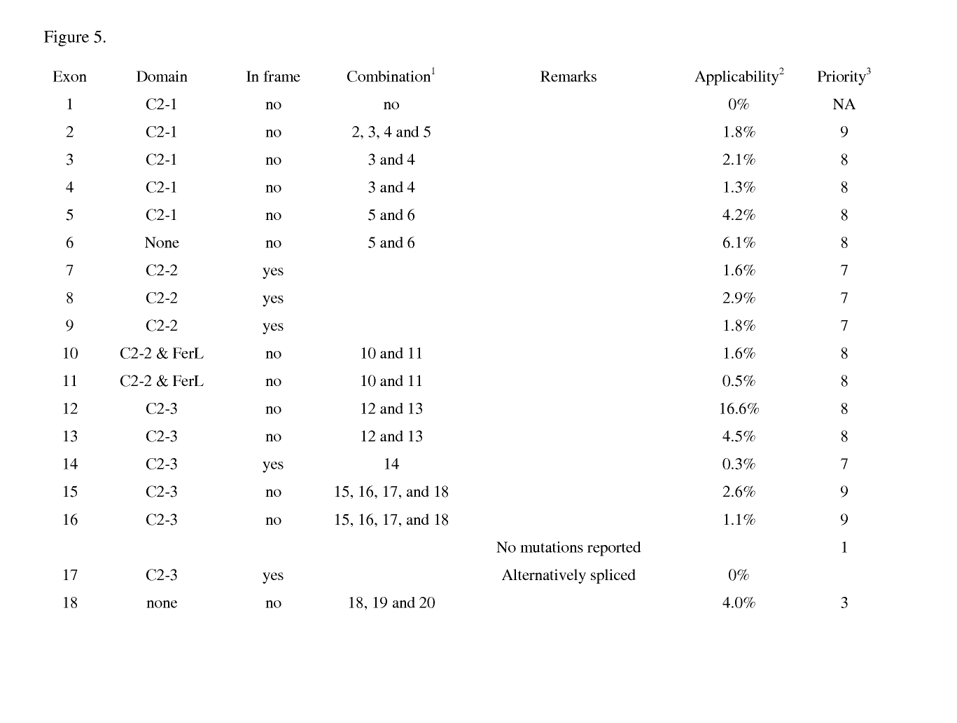

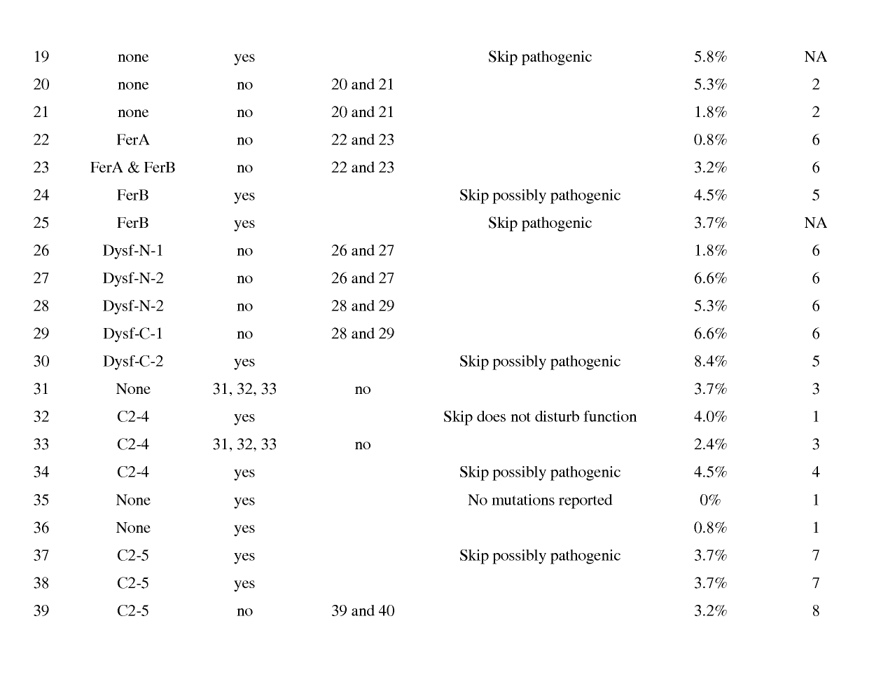

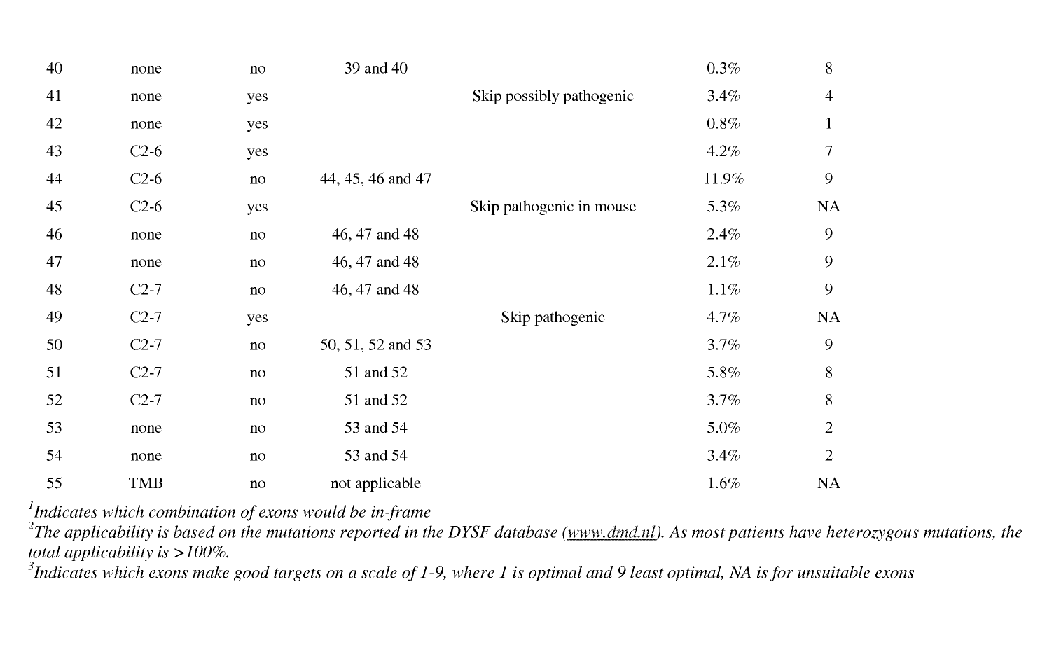

Not every DYSF exon can be skipped without consequence for dysferlin function. First, if the skipped exon is out-of-frame (i.e., the length is not divisible by 3), this will result in a disruption of the open reading frame and a prematurely truncated protein. Thus, either in-frame exons or a combination of out-of-frame exons that together maintain the reading frame, are valid targets (FIGS. 3 and 5).

Secondly, as mentioned, dysferlin contains several domains; and while only limited information is available about their function and essentiality, several things can be learned about these domains from mutations found in patients and animal models. The very mildly affected individual skipping exon 32 suggests that, although exon 32 encodes the fourth C2 domain, a dysferlin without this exon is highly functional (Sinnreich et al., 2006). Thus, apparently the fourth C2 domain is (at least partially) redundant. By contrast, the final C2 domain is likely essential for functionality, since a mouse model SJL/J (SJL-Dysf) with a splice site mutation resulting in the in-frame skipping of exon 45, leading to the omission of the last part of the final C2 domain has a dystrophic phenotype (Vafiadaki et al., 2001, Cloning of the mouse dysferlin gene and genomic characterization of the SJL-Dysf mutation, Neuroreport 12:625-62; Bittner et al., 1999, Dysferlin deletion in SJL mice (SJL-Dysf) defines a natural model for limb girdle muscular dystrophy 2B, Nat. Genet. 23:141-142). The mild patient producing a dysferlin consisting of only the last C2 and the transmembrane domains could suggest that the other four C2 domains are redundant. However, whereas this corresponding mini-dysferlin protein is apparently relatively stable, this does not necessarily hold for all dysferlins with mutations before exon 44, and seems to be exception rather than rule (Therrien et al., 2006).

Thirdly, only internal exons can be skipped, thus, exon 1 and exon 55 are invalid targets. Additional exons that provide invalid targets are in-frame exons 19, 25 and 49 for which splice site mutations resulting in exon-skipping (confirmed by RT-PCR) have been found in LGMD2B and MM patients ((Therrien et al., 2006) and the LOVD DYSF database). Mutations that may affect splicing (i.e., located at or close to the splice sites) have been identified in MM and LGMD2B patients for exons 24, 30, 32, 34, 37 and 41. However, the mutations were found on DNA level and have not been confirmed on RNA level.

Based on this information, exons can be subdivided into suitable and less suitable and impossible candidates (FIG. 5). Even though there are no real mutational hotspots in the DYSF gene, some exons contain more mutations than others and the skipping of these exons would thus be applicable to larger groups of patients (see FIG. 5). Notably, no mutations have been thus far reported for exon 17 and exon 35.

### Example 2

### Analysis of Exon-Skipping

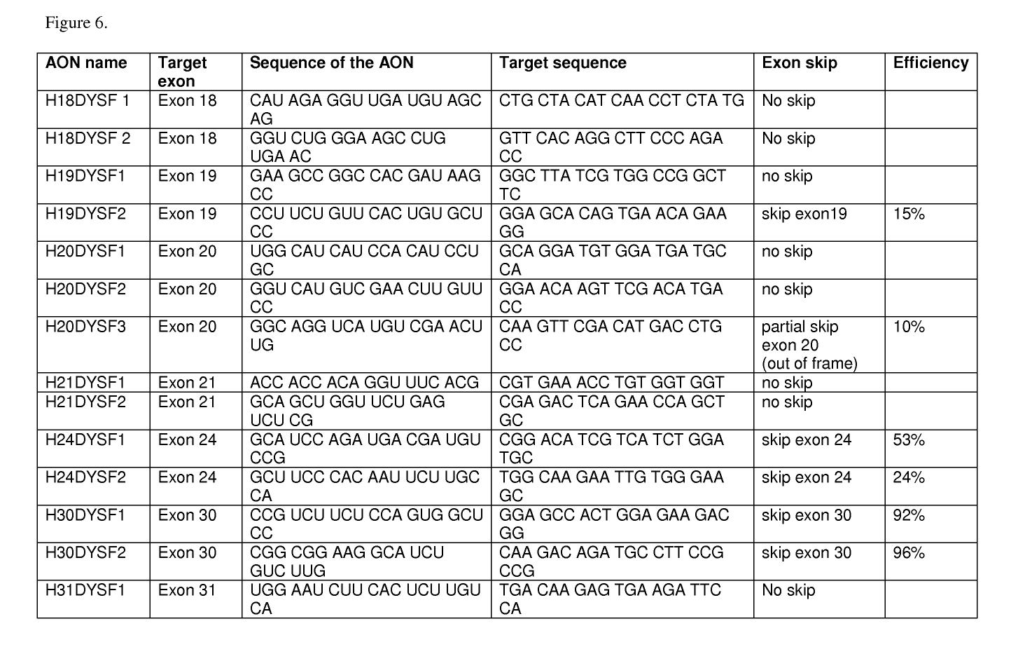

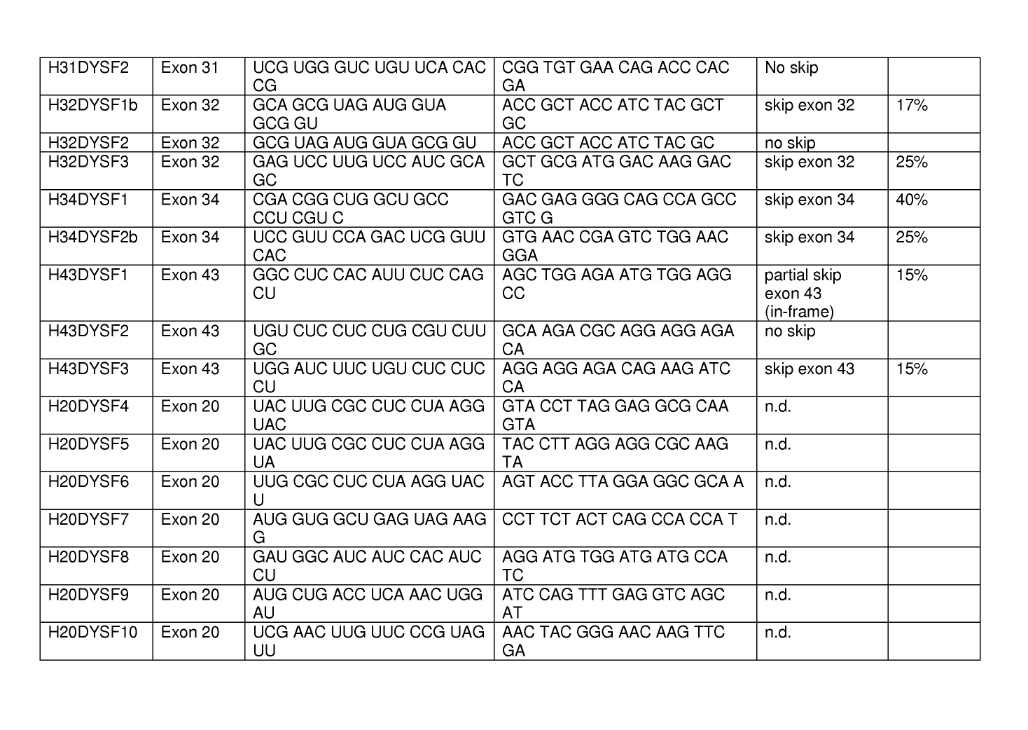

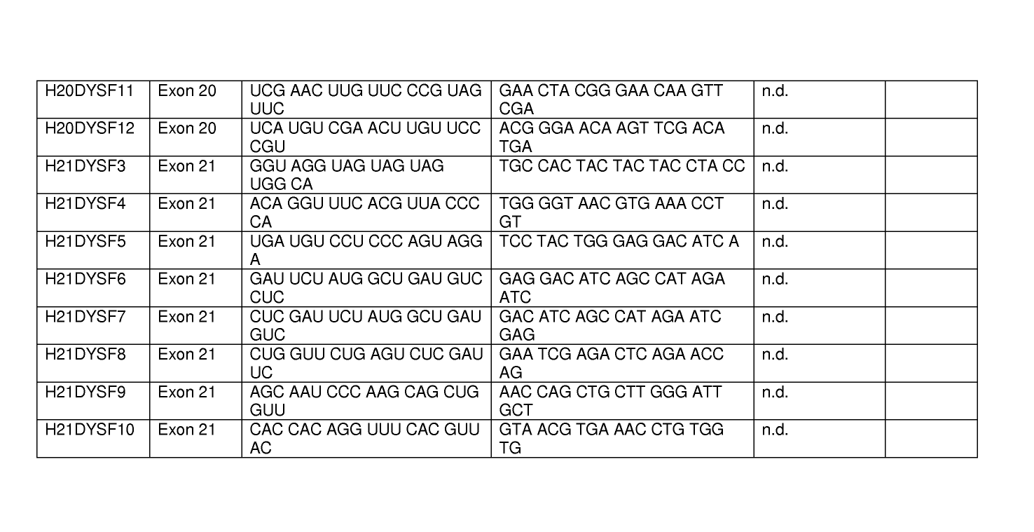

To assess whether exon-skipping can be as achieved for DYSF exons, we designed two oligonucleotides for each exon targeting DYSF exons 18, 19, 21, 24, 30, 31, 32, and 34 and three oligonucleotides targeting exon 20 and 43, using our previously identified oligonucleotide design guidelines (Aartsma-Rus et al., 2005; Aartsma-Rus et al., 2008). Oligonucleotides were transfected in differentiated human control myoblasts. RT-PCR analysis revealed that several oligonucleotides were effective and induced skipping of exons 19, 24, 30, 32, 34, and 43 (FIGS. 4 and 6). Exon-skipping levels varied from 17% (exon 34) to 96% (exon 30). Notably, some spontaneous skipping (alternative splicing) of exon 30 was observed in non-treated cells at low levels (10%).

1. A method for providing a cell with an alternatively spliced dysferlin mRNA, said method comprising:
providing a cell that expresses a dysferlin pre-mRNA with one or more antisense oligonucleotides for skipping exon(s) 32, (2, 3, 4 and 5), (3 and 4), (5 and 6), 7, 8, 9, (10 and 11), (12 and 13), 17, (18, 19 and 20), (20 and 21), (22 and 23), 24, (26 and 27), (28 and 29), 30, (31, 32 and 33), 34, 35, 36, 37, 38, (39 and 40), 41, 42, 43, (44, 45, 46 and 47), (46, 47 and 48), (50, 51, 52 and 53), (51 and 52) or (53 and 54) or a combination thereof; and
allowing splicing of said pre-mRNA.

2. The method of claim 1, wherein one or more antisense oligonucleotides are provided for skipping exon(s) 32, 34, 36, 42, (20 and 21), (53 and 54), (31, 32 and 33), or a combination thereof.

3. The method of claim 1, wherein one or more antisense oligonucleotides are provided for skipping exon(s) 32, 34, 36, 42, or (20 and 21).

4. The method of claim 1, wherein one or more antisense oligonucleotides are provided for skipping exon(s) 32, 34, (20 and 21), 24, 30, 41, 42, (5 and 6), (12 and 13), (26 and 27), (28 and 29), 35, 36, 19, or 43.

5. An oligonucleotide or set of oligonucleotides that is complementary to a dysferlin pre-mRNA and comprises between 15 and 40 nucleotides to induce skipping of exon(s) 32, (2, 3, 4 and 5), (3 and 4), (5 and 6), 7, 8, 9, (10 and 11), (12 and 13), 14, (15, 16, 17 and 18) 17, (18, 19 and 20), (20 and 21), (22 and 23), 24, (26 and 27), (28 and 29), 30, (31, 32 and 33), 34, 35, 36, 37, 38, (39 and 40), 41, 42, 43, (44, 45, 46 and 47), (46, 47 and 48), (50, 51 52 and 53), (51 and 52) or (53 and 54) or a combination thereof.

6. An oligonucleotide or set of oligonucleotides according to claim 5 to induce skipping of exon(s) 32, 34, 36, 42, (20 and 21), (53 and 54), (31, 32 and 33), or a combination thereof.

7. An oligonucleotide or set of oligonucleotides according to claim 5 to induce skipping of exon(s) 32, 34, 36, 42, or (20 and 21).

8. The oligonucleotide or set of oligonucleotides according to claim 5 to induce skipping of exon(s) 32, 34, (20 and 21), 24, 30, 41, 42, (5 and 6), (12 and 13), (26 and 27), (28 and 29), 35, 36, 19, or 43.

9. The oligonucleotide according to claim 5 comprising a sequence selected from the group of SEQ ID NOS:20, 19, and 1-54.

10. The oligonucleotide according to claim 5 comprising a sequence selected from the group of SEQ ID NOS:20, 19, 6, 9, 12-15, 24, 25, 35, and 37.

11. A method for skipping an exon in a pre-mRNA in a cell, said method comprising:
selecting a first oligonucleotide that induces skipping of at least 5% of said exon as assessed by RT-PCR in cells expressing a wild-type form of said pre-mRNA,
further selecting a second oligonucleotide that induces skipping of at least 5% of said exon as assessed by RT-PCR in cells expressing a wild-type form of said pre-mRNA, and
providing said cell with said first and second oligonucleotides.

12. A composition for skipping an exon in a pre-mRNA, the composition comprising two oligonucleotides,
wherein a first oligonucleotide of the two nucleotides induces skipping of at least 5% of said exon as assessed by RT-PCR in cells expressing a wild-type form of said pre-mRNA and
wherein a second oligonucleotide of the two nucleotides induces skipping of at least 5% of said exon as assessed by RT-PCR in cells expressing a wild-type form of said pre-mRNA.

13. A method for skipping an exon in a pre-mRNA, the method comprising:
selecting an oligonucleotide complementary to at least part of a 150 base pair (bp) intron sequence flanking said exon, wherein at least part of the 150 bp intron sequence hybridizes to at least part of said exon; and
providing said oligonucleotide to said cell.

14. An oligonucleotide able to induce skipping of an exon in a pre-mRNA,
wherein the oligonucleotide is complementary to at least part of a 150 base pair (bp) intron sequence flanking said exon and at least part of the 150 bp intron sequence hybridizes to at least part of said exon.

15. A method of selecting an exon-skipping oligonucleotide, the method comprising:
selecting a contiguous region of a pre-mRNA that comprises at least part of the exon to be skipped and at least part of an intronic sequence flanking said exon,
determining the secondary structure of the selected contiguous region, and
designing an oligonucleotide sequence that is complementary to at least part of an intronic sequence predicted to hybridize to at least part of said exon,
wherein said oligonucleotide able to induce skipping of at least 5% of said exon as assessed by RT-PCR in cells expressing a wild-type form of said pre-mRNA.

16. A composition comprising an oligonucleotide selected by the method according to claim 15.

17. A method for skipping an exon in a pre-mRNA in a cell, the method comprising:
selecting an oligonucleotide by the method according to claim 15; and
providing said selected oligonucleotide to said cell.

18. The method according to claim 11, wherein the pre-mRNA is dysferlin.

19. The method according to claim 15, wherein the pre-mRNA is dysferlin.

20. The method according to claim 17, wherein the pre-mRNA is dysferlin.
